SEQUENCE LISTING (40 sequences)

SEQ ID NO: 1 - DNA, 852 bp, Artificial Sequence
  codon-optimized nucleic acid sequence
atgaacggcgaggagcagtactatgccgccacccagctgtacaaggacccctgcgcattccagaggggaccagcacctgagttttctgccagcccccctgcctgtctgtatatgggcagacagccaccacctccaccacctcacccattcccaggcgccctgggcgccctggagcagggcagcccacccgatatctccccatacgaggtgcctccactggcagacgatccagcagtggcccacctgcaccaccacctgcctgcccagctggccctgccacacccacctgcaggacctttcccagagggcgccgagcccggcgtgctggaggagcctaatagggtgcagctgccctttccttggatgaagtctaccaaggcccacgcctggaagggacagtgggcaggaggagcatacgcagccgagcctgaggagaacaagaggacccgcacagcctatacacgcgcccagctgctggagctggagaaggagttcctgtttaataagtatatcagcaggccccggagagtggagctggccgtgatgctgaacctgacagagcgccacatcaagatctggtttcagaataggcgcatgaagtggaagaaggaggaggacaagaagcggggaggaggaaccgcagtgggaggaggcggcgtggccgagcctgagcaggattgcgcagtgacatccggagaggagctgctggccctgccaccccctccaccacctggaggagcagtgccaccagcagcaccagtggccgcccgggagggcagactgcctccaggactgtccgcctctccacagcccagctccgtggcacctcggagaccacaggagcccaggtaa

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 645 bp, Artificial Sequence
  codon-optimized nucleic acid sequence
atgacccctcagccatccggagcaccaacagtgcaggtgacccgcgagacagagcggtccttccccagagcctctgaggacgaggtgacctgccctacatccgccccaccttctccaaccaggacacgcggcaactgcgccgaggcagaggagggaggatgtaggggagcacctagaaagctgagggcaaggagaggcggccggagcagaccaaagtccgagctggccctgagcaagcagaggcgctcccggagaaagaaggccaacgacagggagcgcaatcggatgcacaacctgaatagcgccctggatgcactgaggggcgtgctgcccaccttccctgacgatgccaagctgaccaagatcgagacactgaggtttgcccacaattacatctgggcactgacccagacactgaggatcgcagaccactccctgtatgcactggagccaccagcaccacactgcggagagctgggctctccaggcggcagccccggcgattggggctctctgtacagccccgtgagccaggcaggcagcctgtccccagcagcatctctggaggagagacctggactgctgggagccacattttctgcctgtctgtctcccggcagcctggccttcagcgattttctgtaa

SEQ ID NO: 4 - protein, 214 aa, Homo sapiens
MTPQPSGAPTVQVTRETERSFPRASEDEVTCPTSAPPSPTRTRGNCAEAEEGGCRGAPRKLRARRGGRSRPKSELALSKQRRSRRKKANDRERNRMHNLNSALDALRGVLPTFPDDAKLTKIETLRFAHNYIWALTQTLRIADHSLYALEPPAPHCGELGSPGGSPGDWGSLYSPVSQAGSLSPAASLEERPGLLGATFSACLSPGSLAFSDFL

SEQ ID NO: 5 - DNA, 1062 bp, Artificial Sequence
  codon-optimized nucleic acid sequence
atggcagcagagctggcaatgggagcagagctgcctagctccccactggccatcgagtacgtgaacgacttcgatctgatgaagtttgaggtgaagaaggagccacctgaggcagagaggttctgccacaggctgccaccaggctctctgtctagcacccctctgagcacaccatgctcctctgtgcccagctcccctagcttttgtgccccatcccccggcaccggaggaggaggaggagccggaggaggaggcggctctagccaggccggcggcgcccccggccctccaagcggcggccctggcgccgtgggcggcacctccggcaagcccgccctggaggacctgtactggatgagcggctatcagcaccacctgaacccagaggccctgaatctgacaccagaggatgcagtggaggccctgatcggctccggacaccacggagcccatcacggagcacaccaccctgctgccgccgccgcctacgaggccttccggggaccaggatttgcaggaggaggaggcgccgacgatatgggcgccggccaccaccacggcgcccaccacgccgcccaccatcatcacgccgcccatcatcaccatcatcaccaccaccaccacggaggagcaggacacggaggaggagcaggccaccacgtgcggctggaggagagattctccgacgatcagctggtgtccatgtctgtgcgggagctgaacaggcagctgaggggcttttctaaggaggaagtgatccggctgaagcagaagcggagaaccctgaagaatagaggctatgcccagagctgccggttcaagagagtgcagcagaggcacatcctggagtccgagaagtgtcagctgcagtctcaggtggagcagctgaagctggaggtcggcaggctggcaaaggagcgcgacctgtacaaggagaagtatgagaagctggccggcaggggaggaccaggctctgccggaggagcaggctttccacgcgagcccagcccccctcaggccggccctggcggcgccaagggcacagccgatttctttctgtaa

SEQ ID NO: 6 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 7 - DNA, 1104 bp, Artificial Sequence
  codon-optimized nucleic acid sequence
atgctggcggttggtgcgatggaaggtacccgtcaaagcgcgtttctgctgagcagcccgccgctggcggcgctgcatagcatggcggaaatgaaaaccccgctgtacccggcggcgtacccgccgctgccggcgggtccgccgagcagcagcagcagcagcagcagcagcagcagcccgagcccgccgctgggtacccacaacccgggtggcctgaaaccgccggcgaccggtggcctgagcagcctgggtagcccgccgcaacagctgagcgcggcgaccccgcacggcattaacgacatcctgagccgtccgagcatgccggttgcgagcggtgcggcgctgccgagcgcgagcccgagcggtagcagcagcagcagcagcagcagcgcgagcgcgagcagcgcgagcgcggcggcggcggcggcggcggcggcggcggcggcggcgagcagcccggcgggtctgctggcgggtctgccgcgttttagcagcctgagcccgccgccgccgccgccgggcctgtactttagcccgagcgcggcggcggtggcggcggttggtcgttacccgaaaccgctggcggagctgccgggtcgtaccccgattttctggccgggtgttatgcagagcccgccgtggcgtgacgcgcgtctggcgtgcaccccgcaccaaggtagcattctgctggacaaagatggcaagcgtaaacacacccgtccgaccttcagcggtcaacagatctttgcgctggaaaagaccttcgagcagaccaagtacctggcgggtccggaacgtgcgcgtctggcgtatagcctgggtatgaccgaaagccaggttaaggtttggttccagaaccgtcgtaccaaatggcgtaagaaacacgcggcggagatggcgaccgcgaaaaagaaacaggacagcgagaccgaacgtctgaagggtgcgagcgagaacgaagaagaggacgatgattacaacaaaccgctggacccgaacagcgatgacgagaagatcacccagctgctgaaaaagcacaaaagcagcagcggtggtggtggtggtctgctgctgcacgcgagcgaaccggaaagcagcagctaa

SEQ ID NO: 8 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINDILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 9 - DNA, 822 bp, Artificial Sequence
  codon-optimized nucleic acid sequence
atgagcctgaccaacacaaagaccggcttctccgtgaaggacatcctggatctgccagacacaaacgatgaggagggctccgtggcagagggaccagaggaggagaatgagggcccagagcccgccaagagggcaggaccactgggacagggcgccctggacgccgtgcagtctctgccactgaagaaccccttttacgacagctccgataatccttatacaaggtggctggcatccaccgagggactgcagtactctctgcacggactggcagcaggagcaccacctcaggactctagctccaagtcccctgagccatctgccgatgagagccctgacaacgataaggagacaccaggaggaggaggcgatgcaggcaagaagcggaagcggagagtgctgttctccaaggcccagacctacgagctggagaggcgctttcggcagcagagatatctgtctgcccctgagagggagcacctggcaagcctgatccgcctgacaccaacccaggtgaagatctggttccagaatcaccggtataagatgaagagggccagagccgagaagggcatggaggtgacaccactgccttccccaaggagagtggcagtgcccgtgctggtgagagacggcaagccttgccacgccctgaaggcacaggatctggccgccgccaccttccaggcaggcatcccattttccgcctactctgcccagagcctgcagcacatgcagtacaatgcccagtattctagcgccagcacaccccagtatcctaccgcacacccactggtgcaggcacagcagtggacctggtaa

SEQ ID NO: 10 - protein, 273 aa, Homo sapiens
MSLTNTKTGFSVKDILDLPDTNDEEGSVAEGPEEENEGPEPAKRAGPLGQGALDAVQSLPLKNPFYDSSDNPYTRWLASTEGLQYSLHGLAAGAPPQDSSSKSPEPSADESPDNDKETPGGGGDAGKKRKRRVLFSKAQTYELERRFRQQRYLSAPEREHLASLIRLTPTQVKIWFQNHRYKMKRARAEKGMEVTPLPSPRRVAVPVLVRDGKPCHALKAQDLAAATFQAGIPFSAYSAQSLQHMQYNAQYSSASTPQYPTAHPLVQAQQWTW

SEQ ID NO: 11 - DNA, 1071 bp, Artificial Sequence
  codon-optimized nucleic acid sequence
atgaccaagtcctactctgagagcggcctgatgggagagccacagccacagggaccaccttcttggacagacgagtgcctgagctcccaggacgaggagcacgaggccgataagaaggaggacgatctggaggccatgaacgccgaggaggattccctgcggaatggcggcgaggaggaggatgaggacgaggatctggaggaagaagaggaggaggaggaggaggacgatgaccagaagccaaagcggagaggccccaagaagaagaagatgaccaaggcccgcctggagcggtttaagctgaggcgcatgaaggccaacgcccgggagagaaataggatgcacggcctgaacgccgccctggacaatctgagaaaggtggtgccttgttactccaagacccagaagctgtctaagatcgagacactgagactggccaagaattatatctgggccctgagcgagatcctgaggtccggcaagtctccagatctggtgtccttcgtgcagaccctgtgcaagggcctgtctcagcccaccacaaacctggtggccggctgtctgcagctgaatcctaggacctttctgccagagcagaaccaggacatgccaccacacctgccaacagcatccgcctctttccctgtgcacccatacagctatcagtccccaggactgccttcccctccatacggcacaatggattctagccacgtgtttcacgtgaagccccctccacacgcctattctgccgccctggagcccttctttgagagccctctgaccgactgcacaagcccatccttcgatggccccctgtctccccctctgagcatcaacggcaatttcagctttaagcacgagccctccgccgagttcgagaagaactacgcctttaccatgcactatcctgcagccacactggcaggagcacagtctcacggcagcatcttctccggaaccgcagcacctaggtgtgagatcccaatcgacaatatcatgagctttgattctcacagccaccacgagagagtgatgtccgcccagctgaacgccatcttccacgactaa

SEQ ID NO: 12 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 13 - DNA, 972 bp, Artificial Sequence
  codon-optimized nucleic acid sequence
atggccgccgagctgtctatgggaccagagctgcccaccagccctctggccatggagtacgtgaatgacttcgatctgctgaagtttgacgtgaagaaggagcctctgggaagggcagagagaccaggcaggccctgcacaaggctgcagccagcaggcagcgtgagctccaccccactgtccacaccctgttctagcgtgccttcctctccatccttctctcccaccgagcagaagacacacctggaggatctgtactggatggccagcaactatcagcagatgaatccagaggccctgaacctgaccccagaggacgcagtggaggccctgatcggctcccacccagtgcctcagccactgcagtctttcgatagctttcggggagcacaccatcatcaccaccaccaccacccacaccctcaccacgcctatcctggagcaggagtggcacacgacgagctgggacctcacgcccacccacaccatcaccaccaccaccaggcatccccacctccaagctccgccgcctctcccgcccagcagctgcctacaagccacccaggaccaggacctcacgcaaccgcaagcgccacagccgccggcggcaatggctccgtggaggacagattctctgacgatcagctggtgagcatgtccgtgagggagctgaacaggcacctgaggggctttaccaaggatgaagtgatccgcctgaagcagaagcggagaacactgaagaatcggggctacgcccagtcctgcagatataagagggtgcagcagaagcaccacctggagaacgagaagacccagctgatccagcaggtggagcagctgaagcaggaggtgtcccgcctggccagggagagggacgcctacaaggtgaagtgtgagaagctggccaactctggctttcgggaggccggctctacaagcgattccccctctagccctgagttctttctgtaa

SEQ ID NO: 14 - protein, 323 aa, Homo sapiens
MAAELSMGPELPTSPLAMEYVNDFDLLKFDVKKEPLGRAERPGRPCTRLQPAGSVSSTPLSTPCSSVPSSPSFSPTEQKTHLEDLYWMASNYQQMNPEALNLTPEDAVEALIGSHPVPQPLQSFDSFRGAHHHHHHHHPHPHHAYPGAGVAHDELGPHAHPHHHHHHQASPPPSSAASPAQQLPTSHPGPGPHATASATAAGGNGSVEDRFSDDQLVSMSVRELNRHLRGFTKDEVIRLKQKRRTLKNRGYAQSCRYKRVQQKHHLENEKTQLIQQVEQLKQEVSRLARERDAYKVKCEKLANSGFREAGSTSDSPSSPEFFL

SEQ ID NO: 15 - DNA, 20 bp, Homo sapiens
cgtcttagctgtgcttctgt

SEQ ID NO: 16 - DNA, 22 bp, Homo sapiens
cttggtctgtattgctcctctc

SEQ ID NO: 17 - DNA, 22 bp, Homo sapiens
gggagcaggagtatttcagtag

SEQ ID NO: 18 - DNA, 21 bp, Homo sapiens
gatgtttctgcctgggtatct

SEQ ID NO: 19 - DNA, 20 bp, Homo sapiens
gatggcctccaactggtaaa

SEQ ID NO: 20 - DNA, 22 bp, Homo sapiens
catgtttcaggtgtgcttcttc

SEQ ID NO: 21 - DNA, 22 bp, Homo sapiens
tctctacccttctgctcctatc

SEQ ID NO: 22 - DNA, 22 bp, Homo sapiens
gcatcgagttgagcatgtattc

SEQ ID NO: 23 - DNA, 17 bp, Homo sapiens
gcgcccaatacgaccaa

SEQ ID NO: 24 - DNA, 19 bp, Homo sapiens
ctctctgctcctcctgttc

SEQ ID NO: 25 - DNA, 22 bp, Homo sapiens
gatgcactcagagatgtagtcg

SEQ ID NO: 26 - DNA, 20 bp, Homo sapiens
tgaaggtgggagaaggtgag

SEQ ID NO: 27 - DNA, 22 bp, Homo sapiens
ctccttctctgctctggttatc

SEQ ID NO: 28 - DNA, 21 bp, Homo sapiens
caggttcaggtggatgtagtt

SEQ ID NO: 29 - DNA, 17 bp, Homo sapiens
cttcacgagcccagcca

SEQ ID NO: 30 - DNA, 20 bp, Homo sapiens
atcagaagaggccatcaagc

SEQ ID NO: 31 - DNA, 19 bp, Homo sapiens
atgaggaccacagcctact

SEQ ID NO: 32 - DNA, 22 bp, Homo sapiens
ggaatctggagagatgctgaac

SEQ ID NO: 33 - DNA, 22 bp, Homo sapiens
cagacagcatctacaccatctc

SEQ ID NO: 34 - DNA, 21 bp, Homo sapiens
gtgtaatctccgctgctgtaa

SEQ ID NO: 35 - DNA, 22 bp, Homo sapiens
gacctggcctccaactataatg

SEQ ID NO: 36 - DNA, 22 bp, Homo sapiens
tgtgtaccgagggtaaggatag

SEQ ID NO: 37 - DNA, 21 bp, Homo sapiens
gcatgcccttggagatctatt

SEQ ID NO: 38 - DNA, 20 bp, Homo sapiens
gcagattgggtcggctattt

SEQ ID NO: 39 - DNA, 20 bp, Homo sapiens
gaaagtctgctgggcatttg

SEQ ID NO: 40 - DNA, 22 bp, Homo sapiens
caccatggaccagttaggatag

(19) United States
(12) Patent Application Publication
(10) Pub. No.: US 2020/0190468 A1
(43) Pub. Date: Jun. 18, 2020

(54) GENERATING HUMAN CELLS CAPABLE OF PRODUCING INSULIN IN RESPONSE TO GLUCOSE OR GLP-1

(71) Applicant: Mayo Foundation for Medical Education and Research, Rochester, MN (US)

(72) Inventors: Yasuhiro Ikeda, Rochester, MN (US); Yaxi Zhu, Rochester, MN (US); Aleksey Matveyenko, Rochester, MN (US); Andre Terzic, Rochester, MN (US); Yogish C. Kudva, Rochester, MN (US); Dennis A. Wigle, Rochester, MN (US)

(73) Assignee: Mayo Foundation for Medical Education and Research, Rochester, MN (US)

(21) Appl. No.: 16/607,836

(22) PCT Filed: Apr. 24, 2018
(86) PCT No.: PCT/US2018/029155

§ 371 Date: Oct. 24, 2019

Related U.S. Application Data
(60) Provisional application No. 62/490,348, filed on Apr. 26, 2017.

(51) Int. Cl. C12N 5/0735; C07K 14/605; C07K 14/62; C12N 15/86; C12N 5/074

(52) U.S. Cl. CPC C12N 5/0606; C07K 14/605; C07K 14/62; C12N 2510/00; C12N 5/0696; C12N 2500/34; C12N 2501/335; C12N 15/86

(57) ABSTRACT

This document provides methods and materials related to generating human cells capable of producing insulin in response to glucose or GLP-1. For example, methods and materials for introducing nucleic acid vectors into human stem cells (e.g., human induced pluripotent stem cells) at particular stages of differentiation to create human cells having the ability to produce and secrete human insulin in response to glucose, GLP-1, or both glucose and GLP-1 as measured by a sensitive perifusion assay are provided.

### CROSS-REFERENCE TO RELATED APPLICATIONS

This application claims priority to U.S. Application Ser. No. 62/490,348, filed on Apr. 26, 2017. The disclosure of the prior application is considered part of the disclosure of this application, and is incorporated in its entirety into this application.

### BACKGROUND

### 1. Technical Field

This document relates to methods and materials involved in generating human cells capable of producing insulin in response to glucose or glucagon-like peptide-1 (GLP-1). For example, this document provides methods and materials for making human cells having the ability to produce and secrete human insulin in response to glucose, GLP-1, or both glucose and GLP-1. In some cases, such human cells can be produced as described herein from induced pluripotent stem cells.

### 2. Background Information

Stem cells are characterized by the ability of self-renewal and differentiation into a diverse range of cell types. The two broad types of mammalian stem cells are embryonic stem (ES) cells and adult stem cells. Adult stem cells or progenitor cells replenish specialized cells to repair or maintain regenerative organs. Most adult stem cells are lineage-restricted and generally referred to by their tissue origin, such as adipose-derived stem cells. ES cell lines are derived from the epiblast tissue of the inner cell mass of a blastocyst or early morula stage embryos. ES cells are pluripotent and give rise to derivatives of the three germinal layers, i.e., the ectoderm, endoderm, and mesoderm.

Recently, many different techniques have been developed to create pluripotent stem cells directly from adult cells. Such pluripotent stem cells are generally referred to as induced pluripotent stem cells.

### SUMMARY

This document provides methods and materials related to generating human cells capable of producing insulin in response to glucose or GLP-1. For example, this document provides methods and materials for introducing nucleic acid vectors into human stem cells (e.g., human induced pluripotent stem cells) at particular stages of differentiation to create human cells having the ability to produce and secrete human insulin in response to glucose, GLP-1, or both glucose and GLP-1 as measured by a sensitive islet perifusion assay. Upon high glucose stimulation, cells generated as described herein can exhibit first and second phase insulin secretion, a property unique to functional mature beta cells.

Others may have reported the production of insulin-producing cells with some of those produced cells being reportedly responsive to glucose, however, the production of insulin in response to glucose by those cells wasn't measured using a sensitive perifusion assay, which is a reliable method for assessing real-time, immediate glucose-responsive insulin production. Perifusion allows one to evaluate dynamic (temporal) insulin secretion profiles in response to glucose and other secretagogues. Insulin secretion in vivo occurs in two distinct phases with the first phase (0-5 minutes) corresponding to the release of stored pools of insulin granules and the second phase corresponding to the release of newly formed insulin granules (Curry et al., Endocrinology, 83(3):572-84 (1968); Porte and Pupo, J. Clin. Inv., 48(12):2309-2319 (1969)). Identifying first and second phase temporal insulin profiles is involved in determining proper functionality of stem cell derived islets since lack of first phase insulin secretory response is characteristic of immature and/or dysfunctional beta cells (Dhawan et al., J. Clin. Inv., 125(7):2851-60 (2015); Brunzell et al., J. Clin. Endocrinol. Metab., 42(2):222-9 (1976)). Use of static incubation precludes from detection of first and second phase insulin secretion, which is used to properly evaluate beta cell functionality.

As described herein, human stem cells (e.g., induced pluripotent stem cells) can be obtained and exposed to nucleic acid vectors at particular stages of differentiation to create human cells having the ability to produce and secrete human insulin in response to glucose, GLP-1, or both glucose and GLP-1 as measured by a sensitive perifusion assay. For example, a nucleic acid vector designed to express a pancreatic and duodenal homeobox 1 (PDX1) polypeptide (e.g., a human PDX1 polypeptide) can be introduced into the cells at or during the definitive endoderm stage, a nucleic acid vector designed to express a neurogenin-3 (NGN3 or NEUROG3) polypeptide (e.g., a human NGN3 polypeptide) can be introduced into the cells at or during the pancreatic endoderm stage, and a nucleic acid vector designed to express a MAFA polypeptide (e.g., a human MAFA polypeptide) can be introduced into the cells at or during the primitive beta cell stage. Introducing these vectors at these particular stages of differentiation can result in the formation of human cells having the ability to produce and secrete human insulin in response to glucose, GLP-1, or both glucose and GLP-1 as measured by a sensitive perifusion assay. Such cells can be administered to a human diagnosed with diabetes (e.g., type 1 diabetes) to treat or reduce the symptoms of diabetes and/or diabetes-associated complications, such as chronic kidney disease, cardiovascular diseases, and/or blindness.

In general, one aspect of this document features a population of differentiated cells obtained from pluripotent stem cells (e.g., embryonic stem cells or induced pluripotent stem cells), wherein the cells of the population produce insulin in response to glucose and in response to GLP-1 as measured by a perifusion assay. The cells can be human cells. The cells can lack exogenous nucleic acid. The cells can produce insulin in less than five minutes (e.g., in less than four, three, two, or one minute) in response to at least 16 mM of glucose and in response to at least 100 nM of GLP-1 as measured by a perifusion assay.

In another aspect, this document features a method for obtaining a population of differentiated cells obtained from pluripotent stem cells (e.g., embryonic stem cells or induced pluripotent stem cells), wherein the differentiated cells produce insulin in response to glucose or GLP-1. The method comprises, or consists essentially of, (a) introducing a vector comprising nucleic acid encoding a PDX1 polypeptide into differentiating cells of a pluripotent stem cell population (e.g., an embryonic stem cell population or an induced pluripotent stem cell population) at or during the definitive endoderm stage to form a first cell population, (b) introducing a vector comprising nucleic acid encoding an NGN3 polypeptide into cells of the first cell population at or during the pancreatic endoderm stage to form a second cell population, and (c) introducing a vector comprising nucleic acid encoding a MAFA polypeptide into cells of the second cell population at or during the primitive beta cell stage to form the population of differentiated cells. The induced pluripotent stem cells can be human induced pluripotent stem cells. The embryonic stem cells can be human embryonic stem cells. The differentiated cells produce insulin in response to high glucose or GLP-1 as measured by a perifusion assay. The vector of the step (a), (b), or (c) can be a lentiviral vector. The vector of the step (a), (b), and (c) can be a lentiviral vector. The total number of days starting with the induced pluripotent stem cells to forming the population of differentiated cells can be 20 or less days (e.g., 20, 19, 18, 17, 16, 15, or less days).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1A contains a codon-optimized nucleic acid sequence (SEQ ID NO:1) that encodes a human PDX1 polypeptide, and FIG. 1B contains an amino acid sequence (SEQ ID NO:2) of a human PDX1 polypeptide.

FIG. 2A contains a codon-optimized nucleic acid sequence (SEQ ID NO:3) that encodes a human NGN3 polypeptide, and FIG. 2B contains an amino acid sequence (SEQ ID NO:4) of a human NGN3 polypeptide.

FIG. 3A contains a codon-optimized nucleic acid sequence (SEQ ID NO:5) that encodes a human MAFA polypeptide, and FIG. 3B contains an amino acid sequence (SEQ ID NO:6) of a human MAFA polypeptide.

FIG. 4A contains a codon-optimized nucleic acid sequence (SEQ ID NO:7) that encodes a human NKX6.1 polypeptide, and FIG. 4B contains an amino acid sequence (SEQ ID NO:8) of a human NKX6.1 polypeptide.

FIG. 5A contains a codon-optimized nucleic acid sequence (SEQ ID NO:9) that encodes a human NKX2.2 polypeptide, and FIG. 5B contains an amino acid sequence (SEQ ID NO:10) of a human NKX2.2 polypeptide.

FIG. 6A contains a codon-optimized nucleic acid sequence (SEQ ID NO:11) that encodes a human NEUROD1 polypeptide, and FIG. 6B contains an amino acid sequence (SEQ ID NO:12) of a human NEUROD1 polypeptide.

FIG. 7A contains a codon-optimized nucleic acid sequence (SEQ ID NO:13) that encodes a human MAFB polypeptide, and FIG. 7B contains an amino acid sequence (SEQ ID NO:14) of a human MAFB polypeptide.

FIG. 8 contains photographs of 293T cells infected with lentiviral vectors designed to express codon-optimized PDX1, NKX6.1, NKX2.2, NGN3, NEUROD1, MAFA, and MAFB and stained with DAPI or antibodies directed against PDX1, NKX6.1, NKX2.2, NGN3, NEUROD1, MAFA, or MAFB polypeptides.

FIG. 9 is a graph plotting human C-peptide release (pg/mL/well of 48-well plate) by cells produced from induced pluripotent stem cells via the indicated lentiviral vectors introduced during the definitive endoderm stage (S1) or via the indicated lentiviral vectors introduced during the definitive endoderm stage (S1), the pancreatic endoderm stage (S4), and the primitive beta cell stage (S6). Introduction of vectors designed to increase expression of a single polypeptide at the definitive endoderm stage and no other polypeptides at the pancreatic endoderm stage (S4) or the primitive beta cell stage (S6) did not improve the glucose-responsive insulin secretory capacity of the cells. The combined introduction of a vector designed to express a PDX1 polypeptide during the definitive endoderm stage, of a vector designed to express a NGN3 polypeptide during the pancreatic endoderm stage, and of a vector designed to express a MAFA polypeptide during the primitive beta cell stage resulted in cells capable of producing insulin in response to glucose or GLP-1.

FIG. 10 is a graph plotting glucose-responsive insulin secretion (human C-peptide; pg/mL/minute; 10⁵ cells) by cells formed using a modified Kieffer's protocol or cells formed by introducing a vector designed to express a PDX1 polypeptide during the definitive endoderm stage, introducing a vector designed to express a NGN3 polypeptide during the pancreatic endoderm stage, and introducing a vector designed to express a MAFA polypeptide during the primitive beta cell stage. Results are an average of six independent experiments. Cells formed by introducing PDX1, NGN3, and MAFA polypeptides during the primitive beta cell stage exhibited first and second phase insulin secretion, which confirms proper beta cell functionality.

FIG. 11. Screening of beta-cell transcription factor(s) for improved glucose- and incretin (GLP-1) responsive insulin secretion in psBCs. (A) Schematic diagram of lentiviral vector constructs is shown. (B) 293T cells were infected with lentiviral vectors carrying beta-cell transcription factors. Expression of individual beta-cell factors was verified by immunofluorescent staining using specific antibodies (green). (C) Summary of the 6-stage strategy for iPSCs to psBCs differentiation is shown. (D) Differentiating iPSC progeny cells at S1 were transduced by the EGFP-expressing lentiviral vector, and EGFP expression was monitored by UV microscope at S2, S5 and S6 (left panel). Representative FACS plots for EGFP expression in control and Lenti-EGFP transduced (S1) cells in the end of S6 stage are shown (right panel). Phase, phase contrast. (E) Secretion of human C-peptide by psBCs in response to sequential stimulation of 4 mM, 16 mM and 16 mM+GLP-1 glucose medium was monitored by the perifusion system for an indicated period of time. Different lentiviral vectors were introduced at the indicated stage.

FIG. 12. Secretion of human C-peptide by psBCs in response to sequential stimulation of 4 mM, 16 mM, and 16 mM+GLP-1 glucose medium for an indicated period of time is shown. Different lentiviral vectors were introduced at the indicated stage.

FIG. 13. PNM transduced psBCs are responsive to glucose and incretin (GLP-1) in vitro. (A) Differentiating iPSCs were transduced by PDX1, NEUROG3, and MAFA at S1, S4 and S6, respectively. Secretion of human C-peptide by PNM-transduced (S6-PNM, n=9 biological replicates) and un-transduced control (S6-NUL L, n=8 biological replicates) psBCs, in response to sequential stimulation of 4 mM, 16 mM, 16 mM+GLP-1, 4 mM glucose medium and 30 mM KCL, was monitored by the perifusion system for an indicated period of time. Data represents the mean±S.E.M. of triplicate experiments. (B) Levels of INS and NKX6.1 transcripts in S6 psBCs, transduced with different combinations of PDX1, NEUROG3 and MAFA (n=3 biological replicates and n=2 technical replicates per group), were determined by qRT-PCR and shown as relative to those of Lenti-EGFP-transduced cells. The transcript levels were normalized to glyceraldehyde 3-phosphate dehydrogenase (GAPDH). Data represents the mean±S.E.M.. Compare each group to EGFP transduced cells, *P<0.05, **P<0.01, ***P<0.001; Student's unpaired t-test. (C) Fold changes (FC) of C-peptide levels at the 4-minute time point for 16 mM glucose treatment, 4-minute time point for 16 mM glucose+GLP-1 treatment, 4-minute time point for 4 mM glucose treatment and 6-minute time point for KCL treatment were shown as relative to the levels of the last time point of the first basal 4 mM treatment (n=3 biological replicates per group, n=9 for PNM). Data represents the mean±S.E.M.. Compare Log2FC, *P<0.05, **P<0.01, ***P<0.001; One sample t-test. P+N, PDX1 at S1 and NEUROG3 at S4 transduction; N+M, NEUROG3 at S4 and MAFA at S6; P+M, PDX1 at S1 and MAFA at S6; P+N+M, transduction of 3 factors at stages 1, 4 and 6 (B-C).

FIG. 14. Secretion of human C-peptide by psBCs transduced by different combinations of P/N/M factors in response to sequential stimulation of 4 mM, 16 mM, 16 mM+GLP-1, 4 mM glucose, and 30 mM KCL medium is shown. Data represent the mean ±S.E.M. of the last time point of the first basal 4 mM treatment, 4-minute time point for 16 mM glucose treatment, 16 mM glucose+GLP1 treatment, the second 4 mM glucose treatment and KCL treatment (n=3 biological replicates per group, n=9 for PNM). The key for this figure from top to bottom corresponds to the data bars for each conditions from left to right.

FIG. 15. Induction of NKX6.1, NKX2.2, and NEUROD1 in PNM-transduced psBCs. (A) S6-PNM and S6-NULL psBSc were characterized for key beta-cell/islet protein expression by specific antibodies. Representative immunofluorescent staining images of S6-PNM and S6-NULL psBSc for insulin (INS, green) and C-peptide, NKX6.1, NKX2.2, NEUROD1, glucagon (GCG, red) and somatostatin (SST, light blue) were shown. Nuclei were counterstained by DAPI (dark blue). Scale bars represent in low magnification images, 20 μm, high magnification images, 10 μm. (B) Quantification of colocalization of insulin and NKX6.1, insulin and NKX2.2, or insulin and NEUROD1 in S6-PNM and S6-Null psBCs were shown (in total n=1890 insulin positive cells were counted in S6-PNM, n=770 insulin positive cells were counted in S6-Null). Data represents the mean±S.E.M.. *P<0.05, **P<0.01, ***P<0.001; Student's unpaired t-test. (C) S6-PNM and S6-NULL psBCs were analyzed for insulin (INS) and glucagon (GCG) expression by flow cytometry. Representative FACS plots were shown (n=4 biological replicates in S6-PNM, n=2 biological replicates in S6-NULL). Data represents the mean±S.E.M.. *P<0.05, **P<0.01, ***P<0.001; Student's unpaired t-test. (D) Transmission electron microcopy analysis was performed to image granules within S6-PNM and S6-Null psBCs. Representative insulin granules (red) and glucagon granules (blue) were highlighted by arrows. Scale bars, 200 nm.

FIG. 16 is a representative FACS plot for S6-PNM psBCs for expression of insulin and C-peptide.

FIG. 17. Global gene expression profiles of PN-transduced S5 and PNM-transduced S6 psBCs. (A) Heatmaps of top 30 up-regulated genes for S5-PN and S6-PNM psBCs were shown. Log FC stands for the log2 fold changes of expression level relative to control (n=3 biological replicates per group). (B) Summary of differentially expressed genes related to beta-cell/islet maturation, GSIS, VDCC and KATP channels between S5-Null and S5-PN or S6-Null and S6-PNM psBCs. Log FC stands for the log2 fold change of expression level relative to control (n=3 biological replicates per group). *represents statistically significant difference; Student's unpaired t-test. (C) Quantitative RT-PCR analysis was performed to verify the RNAseq results, using RNA samples from S5-Null, S5-PN, S6-Null and S6-PNM psBCs (n=3 biological replicates and n=2 technical replicates per group). The transcript levels were normalized to GAPDH. Data represents the mean±S.E.M. Different letters represent statistically significant differences between two groups throughout four groups; One-way ANOVA with Tukey test for multiple comparisons.

FIG. 18. Heatmaps of top 30 down-regulated genes for S5-PN and S6-PNM psBCs are shown. Log FC stands for the log2 fold change of expression level relative to control (n=3 biological replicates per group).

FIG. 19. PNM-transduced psBCs show glucose responsive insulin secretion 1-week post transplantation. (A) Experimental design for in vivo psBCs analysis was shown. (B) One week post psBCs transplantation, fasting and 30 minutes (following IPGTT) human C-peptide levels were determined in S6-NULL and S6-PNM psBCs recipient mice, along with STZ-treated, no psBC-transplanted control mice. C-peptide levels from individual mice are shown on scale bar and whisker plots. *P<0.05, **P<0.01, ***P<0.001; Student's paired t-test. (C) Fasting and 30 minutes (following IPGTT) human C-peptide levels in control mice, S6-NULL and S6-PNM psBCs-transplanted mice at 3 weeks, 5 weeks, 9 weeks and 13 weeks post-transplant were shown. C-peptide levels from individual mice are shown on box and whisker plots. *P<0.05, **P<0.01, ***P<0.001; Student's paired t-test. (D) Fasting blood glucose levels after transplantation in control, human beta-cell line, S6-PNM and S6-Null psBC transplanted mice were shown. Data represents the mean±S.E.M. Compare S6-PNM to S6-Null, *P<0.05, **P<0.01, ***P<0.001; Student's unpaired t-test. (E) 13 weeks after transplantation, IPGTT was performed in control, STZ-treated mice (n=3) and STZ-treated mice that received S6-NULL (n=5) and S6-PNM (n=4) psBCs. Data represents the mean±S.E.M.. Letter ‘a’ represents statistically significant difference between control and S6-PNM, letter ‘b’ represents statistically significant difference between control and S6-NULL, letter ‘c’ represents statistically significant difference between S6-NULL and S6-PNM; One-way ANOVA with Tukey test for multiple comparisons. (F) Representative immunofluorescent staining images of S6-PNM and S6-Null psBCs grafts were shown. Insulin (INS, green), glucagon (GCG, red), somatostatin (SST, light blue), were stained with specific antibodies. Nuclei were counterstained by DAPI (blue). Scale bars represent 50 μm. (G) Fluorescence intensity ratios of GCG to INS and SST to INS in images of S6-NULL (n=22 images, 5-6 images per mouse, 4 mice were analyzed) and S6-PNM (n=23 images, 5-6 images per mouse, 4 mice were analyzed) grafts were shown. Data represents the mean±S.E.M.. *P<0.05, **P<0.01, ***P<0.001; Student's unpaired t-test.

FIG. 20. (A) Fasting human C-peptide levels in mice that received S6-NULL and S6-PNM psBCs transplantation is shown. C-peptide levels from individual mice are shown on box and whisker plots. *P<0.05, **P<0.01, ***P<0.001, Student's unpaired t-test. (B) Fasting and 30 min (following IPGTT) human C-peptide levels in control mice, S6-NULL and S6-PNM psBCs and human beta cell line transplanted mice at 3 weeks, 5 weeks, 9 weeks and 13 weeks post-transplant. C-peptide levels from individual mice are shown on box and whisker plots. *P<0.05, **P<0.01, ***P<0.001, Student's paired t-test. (C) IPGTT (left panel) and area under curves (right panel) 7 weeks after transplantations in control mice (n=4), endoC cells transplanted mice (n=3), S6-Null transplanted mice (n=6), S6-PNM transplanted mice (n=7), and 13 weeks after transplantations in control mice (n=4), S6-Null transplanted mice (n=5), S6-PNM transplanted mice (n=4) were shown. Data represents the mean±S.E.M. In left panel, letter ‘a’ represents significant difference between control and S6-PNM, letter ‘b’ represents significant difference between control and S6-NULL, letter ‘c’ represents significant difference between S6-NULL and S6-PNM, letter ‘d’ represents significant difference between control and beta cells, letter ‘d’ represents significant difference between S6-NULL and beta cells, letter ‘e’ represents significant difference between S6-PNM and beta cells; One-way ANOVA with Tukey test for multiple comparisons. In right panel, *P<0.05, **P<0.01, ***P<0.001; Student's unpaired t-test.

FIG. 21. Representative immunofluorescent staining of S6-PNM and S6-Null cell grafts for insulin (INS) and glucagon (GCG) is shown. Nuclear DAPI staining is shown in blue. Scale bars represent 100 μm.

### DETAILED DESCRIPTION

This document provides methods and materials related to generating human cells capable of producing insulin in response to glucose or GLP-1. For example, this document provides methods and materials for introducing nucleic acid vectors into human stem cells (e.g., human induced pluripotent stem cells or embryonic stem cells) at particular stages of differentiation to create human cells having the ability to produce and secrete human insulin in response to glucose, GLP-1, or both glucose and GLP-1 as measured by a sensitive perifusion assay. This document also provides cells (e.g., human cells) that underwent guided differentiation from induced pluripotent stem cells, compositions containing cells that underwent guided differentiation from induced pluripotent stem cells, and methods for using cells that underwent guided differentiation from induced pluripotent stem cells (e.g., methods for using such cells to treat diabetes).

As described herein, the methods and materials provided herein can be used to guide the differentiation of pluripotent stem cells (e.g., human induced pluripotent stem cells or embryonic stem cells) into more specialized cells that have the ability to produce insulin in response to glucose or GLP-1 as measured by a sensitive perifusion assay. Such a perifusion assay can be used to assess dynamic insulin secretion of pluripotent stem cell-derived insulin-producing cells in vitro and can be performed as described elsewhere (e.g., Song et al., J. Clin. Endocrinol. Metab., 87(1):213-21 (2002)). Briefly, basal glucose perfusate (4 mM glucose) can be delivered from 0-30 minutes followed by 30 minutes of high glucose perfusate (16 mM glucose), 30 minutes of high glucose and GLP-1 perfusate (16 mM glucose+GLP-1), and 10 minutes of a non-specific insulin secretagogue (KCl) to the perifusion chambers containing stem cell-derived cells, and the effluent can be collected in one minute intervals for subsequent determination of insulin concentrations. Human C-peptide (insulin byproduct) concentrations can be measured by ELISA. Minute-by-minute insulin/C-peptide concentration profiles can be analyzed to determine rates of basal and glucose-stimulated insulin secretion.

Any appropriate embryonic stem cell population or induced pluripotent stem cell population can be used to make cells having the ability to produce insulin in response to glucose or GLP-1. For example, induced pluripotent stem cell population produced as described elsewhere (Fusaki et al., Proc. Jpn. Acad., Ser. B, 85:348-362 (2009); Yu et al., Science, 324(5928):797-801 (2009); VandenDriessche et al., Blood, 114(8):1461-8 (2009); Subramanyam et al., Nature Biotechnology, 29(5):443-8 (2011); Anokye-Danso et al., Cell. Stem Cell, 8(4):376-88 (2011)) can be used as described herein.

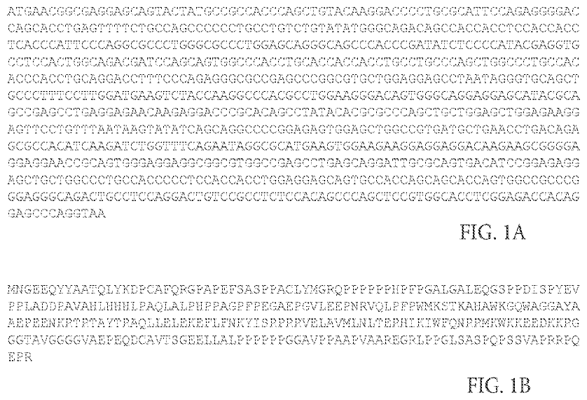

Once embryonic stem cells or induced pluripotent stem cells are obtained, the cells can be processed through a differentiation procedure that involves multiple steps (e.g., six steps). In some cases, throughout the process, the same basal medium (MCDB 131 medium supplemented with Glutamate, antibiotics (e.g., penicillin and streptomycin), 10 mM glucose, and 1.5 g/L sodium bicarbonate) can be used. During step 1, the cells can be cultured to generate endoderm cells. This step 1 can last about three days. In general, the cells are cultured in basal medium that can be further supplemented with serum (e.g., such as from about 0.2 percent to about 2 percent of FCS), activin A (e.g., from about 20 to about 500 ng/mL), and a GSK3b inhibitor (e.g., from about 0.5 to about 20 μM of CHIR-99021; SelleckChem, S2924). Other ingredients that optionally can be used include, without limitation, bovine serum albumin (BSA), GDF8, sodium bicarbonate, and glucose. At some point during step 1 (e.g., during day 2 or day 3), a vector (e.g., a lentiviral vector) designed to express a PDX1 polypeptide (e.g., a human PDX1 polypeptide) can be introduced into the cells. For example, the cells can be infected with a lentiviral vector designed to express a human PDX1 polypeptide. A PDX1 polypeptide can have the amino acid sequence set forth in GenBank® Accession Number NP_000200.1 (e.g., GI No. 136125) or as set forth in FIG. 1B. In some cases, a codon-optimized nucleic acid sequence that encode a PDX1 polypeptide (see, e.g., FIG. 1A) can be used to create a vector for expressing a PDX1 polypeptide. Endoderm cells can be confirmed by identifying the following nuclear expression profile: FOXA2 and CXCR4.

During step 2, the cells can be cultured to generate primitive gut tube-like cells. This step 2 can last about two days. In general, the cells are cultured in basal medium that can be supplemented with serum (e.g., from about 0.2 percent to about 2 percent FCS), ascorbic acid (e.g., from about 0.1 to about 1.0 mM), and FGF7 (e.g., from about 10 to about 200 ng/mL). Other ingredients that optionally can be used include, without limitation, BSA, sodium bicarbonate, and glucose. In some cases, primitive gut tube-like cells can be identified using the following nuclear expression profile: HNF1 and HNF4.

During step 3, the cells can be cultured to generate posterior foregut-like cells. This step 3 can last about two days. In general, the cells are cultured in basal medium that can be supplemented with a sodium bicarbonate solution (e.g., from about 0.5 to about 5 g/L), FCS (e.g., from about 0.5 to about 5 percent), ascorbic acid (e.g., from about 0.05 to about 1.0 mM), FGF7 (e.g., from about 10 to about 50 ng/mL), SANT-1 (e.g., from about 0.05 to about 1.0 μM), retinoic acid (e.g., from about 0.2 to about 5.0 μM), an ALK2/3 inhibitor (e.g., from about 10 to about 500 nM of an ALK2/3 inhibitor such as LDN193189), Insulin-Transferrin-Selenium-Ethanolamine (ITS-X; Life Technologies, Cat #51500056; e.g., from about 0.2 to about 2 percent), and a PKC activator (e.g., from about 40 to about 1000 nM of a PKC activator such as TPB). Other ingredients that optionally can be used include, without limitation, BSA, glucose, and Indolactam V. In some cases, posterior foregut-like cells can be identified using the following nuclear expression profile: PDX1.

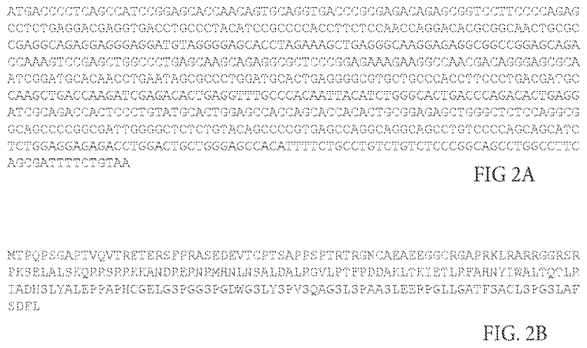

During step 4, the cells can be cultured to generate pancreatic endoderm cells. This step 4 can last about three days. In general, the cells are cultured in basal medium that can be supplemented with sodium bicarbonate solution (e.g., from about 0.5 to about 5.0 g/L), FCS (e.g., from about 0.5 to about 5.0 percent), ascorbic acid (e.g., from about 0.05 to about 1.0 mM), FGF7 (e.g., from about 0.4 to about 10 ng/mL), SANT-1 (e.g., from about 0.05 to about 1.0 μM), retinoic acid (e.g., from about 0.01 to about 0.5 μM), an ALK2/3 inhibitor (e.g., from about 40 to about 1000 nM of an ALK2/3 inhibitor such as LDN193189), ITS-X (e.g., from about 0.2 to about 2 percent), and a PKC activator (e.g., from about 40 to about 1000 nM of a PKC activator such as TPB). Other ingredients that optionally can be used include, without limitation, BSA, glucose, and Indolactam V. At some point during the last half of step 3 and the first half of step 4, a vector (e.g., a lentiviral vector) designed to express a NGN3 polypeptide (e.g., a human NGN3 polypeptide) can be introduced into the cells. For example, the cells can be infected with a lentiviral vector designed to express a human NGN3 polypeptide. A NGN3 polypeptide can have the amino acid sequence set forth in GenBank® Accession Number NP_066279.2 (e.g., GI No. 1292057) or as set forth in FIG. 2B. In some cases, a codon-optimized nucleic acid sequence that encode a NGN3 polypeptide (see, e.g., FIG. 2A) can be used to create a vector for expressing a NGN3 polypeptide. Pancreatic endoderm cells can be confirmed by identifying the following nuclear expression profile: PDX1.

During step 5, the cells can be cultured to generate pancreatic endocrine progenitor cells. This step 5 can last about three days. In general, the cells are cultured in basal medium that can be supplemented with sodium bicarbonate solution (e.g., from about 0.5 to about 5.0 g/L), glucose (e.g., from about 1 to about 10 μL/mL of 45% glucose solution), FCS (e.g., from about 0.5 to about 5.0%), SANT-1 (e.g., from about 0.05 to about 1.0 μM), retinoic acid (e.g., from about 0.01 to about 0.5 μM), an ALK5 inhibitor II (e.g., Enzo Life Sciences, Cat # ALX-270-445, from about 1 to about 50 μM), an ALK2/3 inhibitor (e.g., from about 10 to about 500 nM of an ALK2/3 inhibitor such as LDN193189), a thyroid hormone T3 (e.g., from about 0.2 to about 5 μM of a thyroid hormone such as T3 3,3′,5-Triiodo-L-thyronine sodium salt, Sigma, T6397), zinc sulfate (e.g., from about 2 to about 50 μM), heparin (e.g., from about 2 to about 50 μg/mL), and ITS-X (e.g., from about 0.2 to about 2.0 percent). Other ingredients that optionally can be used include, without limitation, BSA, betacellulin, GLP-1, exendin-4, Trolox, N-acetyl cysteine, AXL inhibitor, EGF, HGF, and CNTF. Pancreatic endocrine progenitor cells can be identified using the following nuclear expression profile: PDX1 and NEUROD1.

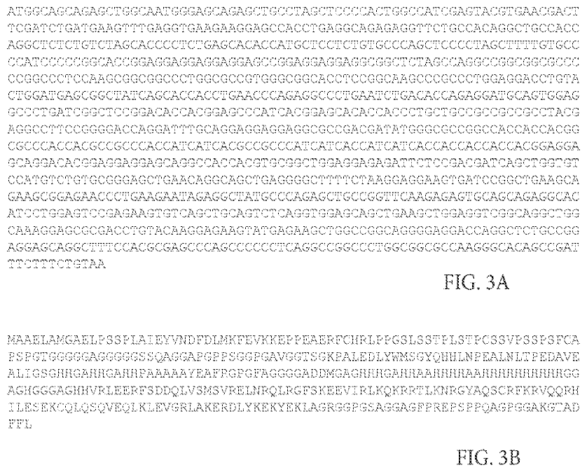

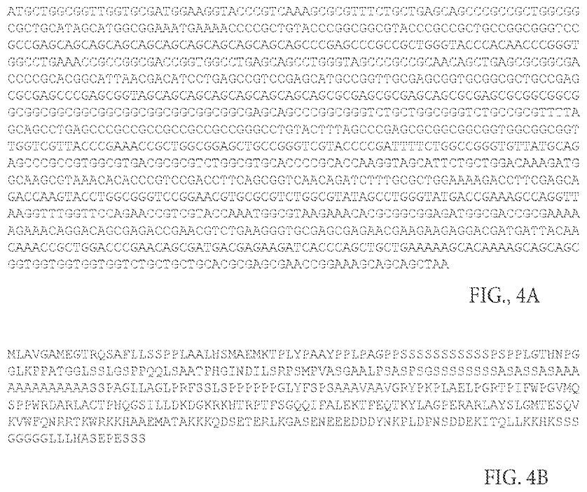

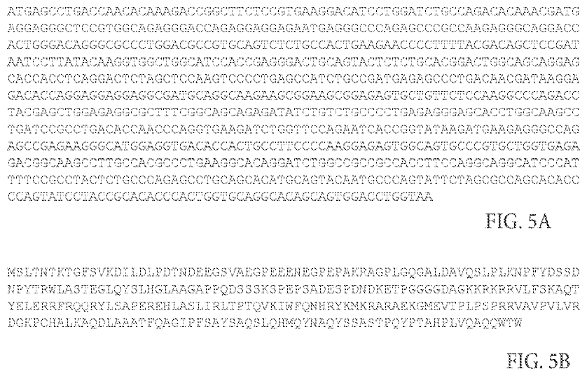

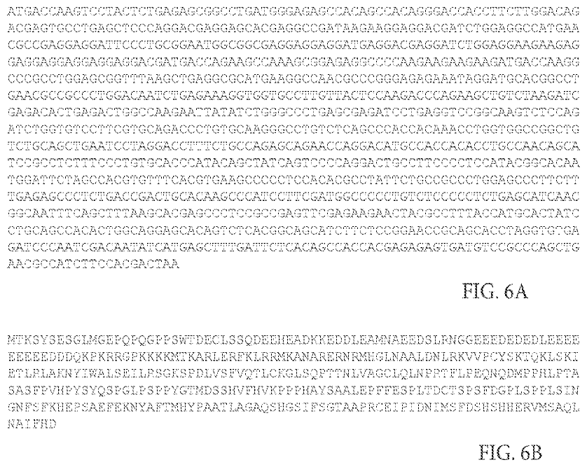

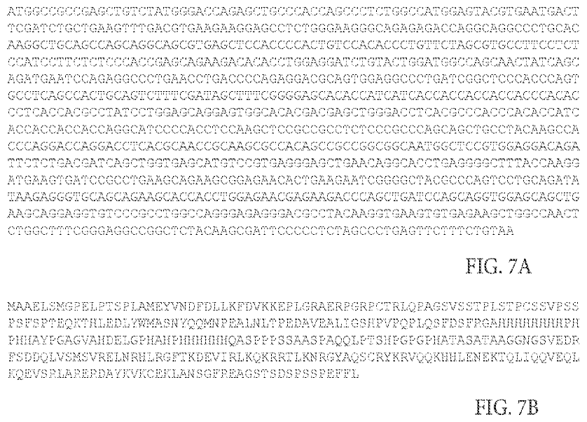

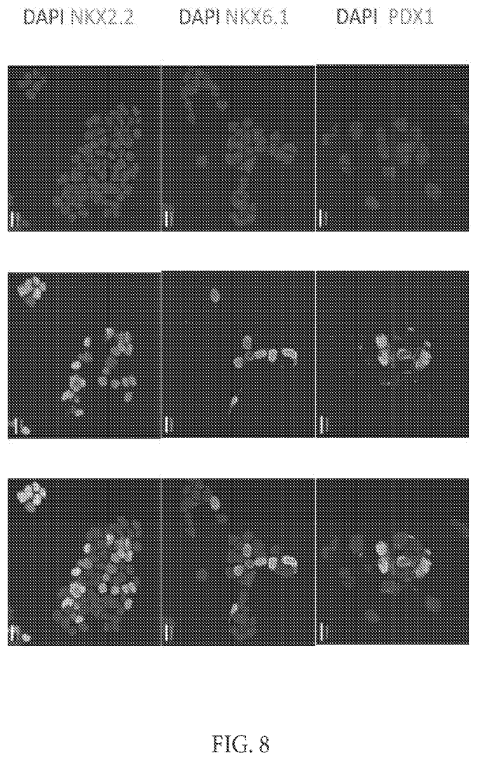

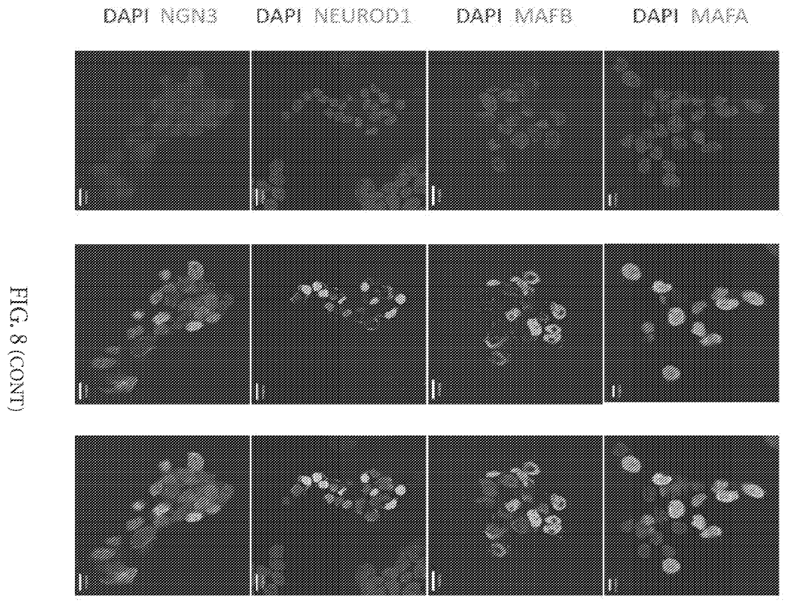

During step 6, the cells can be cultured to generate glucose-responsive/insulin-positive cells (i.e., mature beta cells) as well as glucagon (GCG)-secreting alpha cells, another component of human pancreatic islets controlling glucose homeostasis. This step 6 can last about three to ten days. In general, the cells are cultured in basal medium that can be supplemented with sodium bicarbonate solution (e.g., from about 0.5 to about 5.0 g/L), glucose (e.g., from about 1 to about 10 μL/mL of 45% glucose solution), FCS (e.g., from about 0.5 to about 5 percent), thyroid hormone T3 (e.g., from about 0.2 to about 5 μM), an ALK5 inhibitor II (e.g., from about 1 to about 50 μM), zinc sulfate (e.g., from about 2 to about 50 μM), an ALK2/3 inhibitor (e.g., from about 10 to about 500 nM of an ALK2/3 inhibitor such as LDN193189), ITS-X (e.g., from about 0.2 to about 2.0 percent), heparin (e.g., from about 2.0 to about 50 μg/mL), and gamma secretase inhibitor XX (e.g., from about 20 to about 500 nM). Other ingredients that optionally can be used include, without limitation, BSA, betacellulin, GLP-1, exendin-4, Trolox, N-acetyl cysteine, AXL inhibitor, EGF, HGF, and CNTF. At some point between the end of step 5 and during step 6, a vector (e.g., a lentiviral vector) designed to express a MAFA polypeptide (e.g., a human MAFA polypeptide) can be introduced into the cells. For example, the cells can be infected with a lentiviral vector designed to express a human MAFA polypeptide. A MAFA polypeptide can have the amino acid sequence set forth in GenBank Accession Number NP_963883.2 (e.g., GI No. 912661) or as set forth in FIG. 3B. In some cases, a codon-optimized nucleic acid sequence that encode a MAFA polypeptide (see, e.g., FIG. 3A) can be used to create a vector for expressing a MAFA polypeptide. Mature beta cells can be confirmed by identifying the following gene expression profile: insulin, IAPP, and GCG.

In some cases, a non-integrating viral vector can be used to introduce a nucleic acid designed to express a PDX1 polypeptide, an NGN3 polypeptide, or a MAFA polypeptide into cells as described herein. Examples of non-integrating viral vectors that can be used as described herein include, without limitation, Sendai viral vectors, measles viral vectors, parainfluenza viral vectors, adenoviral vectors, adeno-associated virus vectors, and non-integrating lentiviral vectors with mutated integrase.

Any appropriate method can be used to introduce nucleic acid (e.g., a nucleic acid vector) designed to express a PDX1 polypeptide, an NGN3 polypeptide, or a MAFA polypeptide into a cell. For example, a nucleic acid vector encoding a PDX1 polypeptide, an NGN3 polypeptide, or a MAFA polypeptide can be transferred to the cells using recombinant viruses that can infect cells, or liposomes or other non-viral methods such as electroporation, microinjection, transposons, phage integrases, or calcium phosphate precipitation, that are capable of delivering nucleic acids to cells. The exogenous nucleic acid that is delivered typically is part of a vector in which a regulatory element such as a promoter is operably linked to the nucleic acid of interest. The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus.

Additional regulatory elements that may be useful in vectors, include, but are not limited to, polyadenylation sequences, translation control sequences (e.g., an internal ribosome entry segment, IRES), enhancers, or introns. Such elements may not be necessary, although they can increase expression by affecting transcription, stability of the mRNA, translational efficiency, or the like. Such elements can be included in a nucleic acid construct as desired to obtain optimal expression of the nucleic acids in the cells. Sufficient expression, however, can sometimes be obtained without such additional elements.

Vectors also can include other elements. For example, a vector can include a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), sodium iodine symporter (NIS), and xanthin-guanine phosphoribosyltransferase (XGPRT). Such markers are useful for selecting stable transformants in culture.

Any appropriate non-viral vectors can be used to introduce nucleic acid encoding a PDX1 polypeptide, an NGN3 polypeptide, or a MAFA polypeptide into cells. Examples of non-viral vectors include, without limitation, vectors based on plasmid DNA or RNA, retroelement, transposon, and episomal vectors. Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. See, Felgner et al., J. Biol. Chem., 269:2550-2561 (1994). High efficiency liposomes are commercially available. See, for example, SuperFect® from Qiagen (Valencia, Calif.).

In some cases, the culturing steps described herein can be performed using culture conditions that do not involve the use of serum or feeder cells. For example, steps 1-6 can involve culturing the cells in medium lacking serum (e.g., human or non-human serum) and lacking feeder cells (e.g., human or non-human feeder cells). In such cases, BSA and ITS-X can be used.

In some cases, when non-integrated vectors or nucleic acid transfection methods are used to introduce PDX1, NGN3 and/or MAFA, after the cells capable of producing insulin in response to glucose or GLP-1 are formed, the cells can be maintained in culture such that they become devoid of any introduced exogenous nucleic acid.

In some cases, the cells capable of producing insulin in response to glucose or GLP-1 as described herein can be administered to a human diagnosed with diabetes (e.g., type 1 diabetes) to treat or reduce the symptoms of diabetes. For example, about 1×10⁹ to about 1×10¹⁰ cells capable of producing insulin in response to glucose or GLP-1 as described herein can be administered into a portal vein, liver, or subcutaneous space of a human with diabetes via infusion or after encapsulation into a transplantable immune-protecting device. Once these cells are administered to the human, the severity of the diabetes symptoms can become less severe. In some cases, the stem cell-derived glucagon-producing cells can prevent hypoglycemia induced by exogenous insulin therapy in patients with diabetes.

The invention will be further described in the following examples, which do not limit the scope of the invention described in the claims.

### EXAMPLES

### Example 1—Generating Differentiated Cells from iPSCs

The following was performed to produce human cells having the ability to produce and secrete human insulin in response to glucose or GLP-1. iPSCs were obtained as described elsewhere (Hu et al., Blood, 117(14):e109-19 (2011)). Basal medium was created by supplementing MCDB131 medium (500 mL) with 1× Glutamax, 1× Antibiotics (P&S), 900 μL of D-glucose solution (45%, Sigma G8769), and 10 mL of sodium bicarbonate solution (7.5%, Sigma S8761).

Step 1 involved generating definitive endoderm cells, which lasted 3 days. Before starting differentiation, the iPSCs were plated on Matrigel-coated 12- or 6-well plates and maintained for few days until reaching 50-80% confluence. Some spaces were observed between iPSC colonies. On day 1, the cells were first rinsed with DPBS without Mg2+ and Ca2+ and then cultured in the basal medium, supplemented with 0.5% FCS, 100 ng/mL Activin A, and 3 μM of CHIR-99021 (GSK3b inhibitor, SelleckChem, S2924) (2 mL each for a well of a 6-well-plate, 1 mL for a 12-well-plate). The cells were incubated for one day. On days 2-3, the culture supernatants were removed, and the cells were incubated with the basal medium with 0.5% FCS and 100 ng/mL Activin A (both day 2 and day 3). Also, on day 2, the cells were infected with a lentiviral vector designed to express a human PDX1 polypeptide.

Step 2 involved generating primitive gut tube-like cells, which lasted 2 days. Briefly, on day 4 of differentiation, the cells were rinsed with DPBS without Mg2+ and Ca2+ once, and the culture supernatants were replaced with basal medium with 0.5% FCS, 0.25 mM ascorbic acid (AA; Sigma, Cat # A4544), and 50 ng/mL of FGF7 (R&D Systems). On day 5, the cells were fed fresh medium with the same ingredients.

Step 3 involved generating posterior foregut-like cells, which lasted 2 days. On day 6, the culture supernatants were replaced with basal medium further supplemented with 14 μL/mL of 7.5% sodium bicarbonate solution, 2% FCS, 0.25 mM ascorbic acid (AA), 50 ng/mL of FGF7, 0.25 μM SANT-1 (Shh inhibitor, Sigma, Cat # S4572), 1 μM retinoic acid (RA; Sigma, Cat # R2625), 100 nM LDN193189 (LDN; ALK2/3 inhibitor, Stemgent, Cat #04-0019), 1:200 ITS-X (Life technologies, Cat #51500056), and 100 nM TPB (PKC activator, also called alpha amyloid protein modulator). On day 7, the cells were fed fresh medium with the same ingredients.

Step 4 involved generating pancreatic endoderm cells, which lasted 3 days. On day 8, the culture supernatants were replaced with basal medium further supplemented with 14 μL/mL of 7.5% sodium bicarbonate solution, 2% FCS, 0.25 mM AA, 2 ng/mL of FGF7, 0.25 μM SANT-1, 0.1 μM RA, 200 nM LDN, 1:200 ITS-X, and 100 nM TPB. In addition, the cells were infected with a lentiviral vector designed to express a human NGN3 polypeptide either on day 7 (end of Step 3) or on day 8 (beginning of Step 4). On days 9 and 10, the cells were fed fresh medium with the same ingredients.

Step 5 involved generating pancreatic endocrine progenitor cells, which lasted 3 days. On day 11, the cells were cultured in basal medium further supplemented with 14 μL/mL of 7.5% sodium bicarbonate solution, 4 μL/mL of 45% glucose solution, 2% FCS, 0.25 μM SANT-1, 10 μM ALK5 inhibitor II (Enzo Life Sciences, Cat # ALX-270-445), 0.05 μM RA, 1 μM thyroid hormone T3 (3,3′,5-Triiodo-L-thyronine sodium salt, Sigma, T6397), 100 nM LDN, 1:200 ITS-X, 10 μM zinc sulfate (Sigma, Z0251), and 10 μg/mL of heparin (Sigma, H3149). On days 12 and 13, the cells were fed fresh medium with the same ingredients.

Step 6 involved generating NKX6.1⁺/insulin⁺ cells, which lasted 7 days. On day 14, the cells were cultured in basal medium further supplemented with 14 μL/mL of 7.5% sodium bicarbonate solution, 4 μL/mL of 45% glucose solution, 2% FCS, 1 μM T3, 10 μM ALK5 inhibitor II, 10 μM zinc sulfate, 100 nM LDN, 1:200 ITS-X, 10 μg/mL of heparin, and 100 nM gamma secretase inhibitor XX (EMD MilliPore, Cat #565789). The cells were infected with a lentiviral vector designed to express a human MAFA polypeptide on day 16. In addition, the cells were fed fresh medium with the same ingredients each of the seven days of Step 6.

The cells produced using these six steps were evaluated for insulin expression (via C-peptide release) in response to glucose (4 mM or 16 mM), GLP-1 (100 nM), or KCL (30 mM potassium chloride, a powerful membrane-depolarizing agent to induce insulin release). In addition, cells produced using the identical protocol with the sole exception of only one infection during step 1 with a lentiviral vector designed to express one of the following seven polypeptides were assessed for comparison: PDX1, NKX6.1, NKX2.2, NGN3, MAFA, MAFB, or NEUROD1.

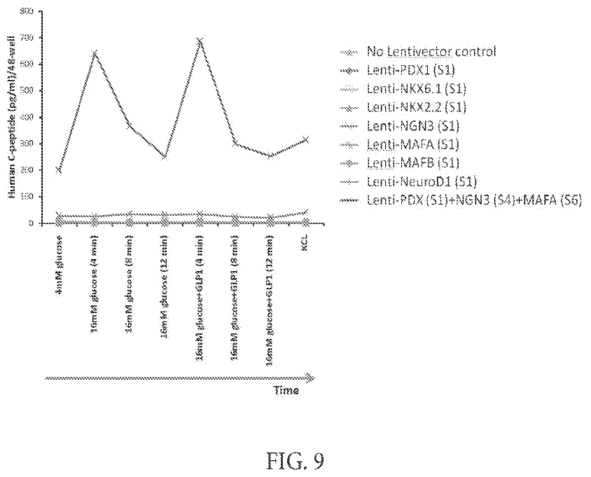

The combined introduction of a vector designed to express a PDX1 polypeptide during the definitive endoderm stage (Step 1), of a vector designed to express a NGN3 polypeptide during the pancreatic endoderm stage (Step 4), and of a vector designed to express a MAFA polypeptide during the primitive beta cell stage (Step 6) resulted in cells capable of producing insulin in response to glucose or GLP-1 (FIG. 9). The cells only receiving an infection with one type of lentiviral vector did not exhibit insulin production in response to glucose or GLP-1 (FIG. 9).

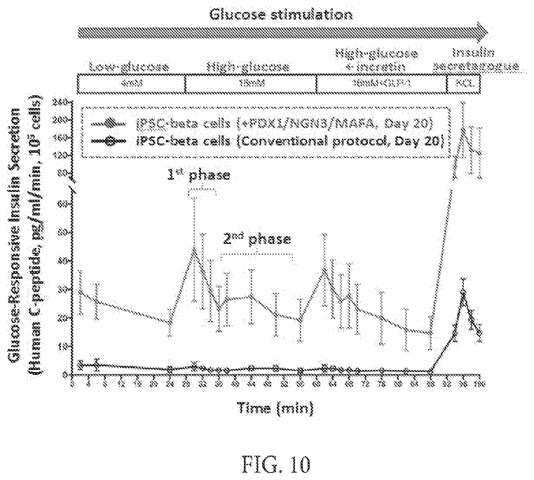

The iPSC differentiation process was repeated with lentiviral introductions of PDX1, NGN3, and MAFA at Steps 1, 4, and 6, respectively. As a control, iPSCs were differentiated without lentiviral vector infection. Upon perifusion assays, the temporal C-peptide secretion patterns were determined using averages of six independent samples (FIG. 10).

These results demonstrate that human cells having the ability to produce insulin in response to glucose, GLP-1, or both can be produced from stem cells (iPSCs) by introducing a nucleic acid vector designed to express a PDX1 polypeptide into the cells at or during the definitive endoderm stage, by introducing a nucleic acid vector designed to express a NGN3 polypeptide into the cells at or during the pancreatic endoderm stage, and by introducing a nucleic acid vector designed to express a MAFA polypeptide into the cells at or during the primitive beta cell stage. Such insulin production can be insulin production as measured by a sensitive perifusion assay.

### Example 2—Targeted Derivation of Organotypic Glucose- and GLP-1-Responsive Beta Cells Prior to Transplantation into Diabetic Recipients

Some of the results provided in this Example 2 were presented in Example 1 and are being represented in this Example 2.

### Cells

293T cells were cultured in DMEM medium supplemented with 10% fetal calf serum and antibiotics. Undifferentiated iPSC line IISH2i-BM9 (WiCell, Madison, Wis.) at passage 10-30 was cultured on Matrigel (Corning, Corning, N.Y., #354277) -coated plates in mTeSR1 medium (StemCell Technologies, Vancouver, Canada, #05850). Cultures were fed every day with mTeSR1 medium.

### Lentiviral Vectors

Lentiviral vector genome plasmid, pSIN-CSGW-PKG-puro, which supports EGFP expression and puromycin selection, was obtained from Dr. Paul Lehner (Cambridge Institute for Medical Research). Codon-optimized ORF sequences for beta-cell factors, including PDX1, NEUROG3, NKX2.2, NKX6.1, NEUROD1, MAFA, MAFB and ESRRG, were designed and synthesized (GenScript, Piscataway, N.J.), and cloned into the place of the EGFP gene in pSIN-CSGW-PKG-puro with the unique BamHI and XhoI sites. Resulting vector plasmids were designated as pLenti-PDX1, pLenti-NEUROG3, pLenti-NKX2.2, pLenti-NKX6.1, pLenti-NEUROD1, pLenti-MAFA, pLenti-MAFB and pLenti-ESRRG, respectively. The internal spleen focus-forming virus (SFFV) promoter drives the expression of beta-cell factors. Lentiviral vectors were produced by plasmid transfection in 293T cells as described elsewhere (Tonne et al., Clin. Chem., 57:864-873 (2011)), concentrated by ultracentrifugation and re-suspended in phosphate buffered saline (PBS). Lentiviral titers were determined by puromycin selection.

### Guided Differentiation and Stepwise Lentiviral Vector Transduction

Guided differentiation was initiated 48 hours following seeding, with a 60-80% starting confluency with some spaces between iPSCs colonies. The basal medium was prepared by supplementing MCDB131 medium (Thermo Fisher Scientific, Waltham, Mass., #10372019) with 1× Glutamax (Thermo Fisher Scientific, #35050061), 50 U/mL Penicillin, 50 μg/mL Streptomycin, 0.02% D-Glucose solution (45%, Sigma-Aldrich, St. Louis, Mo., # G8769) and 2% Sodium Bicarbonate Solution (7.5%, Sigma-Aldrich, # S8761). Step 1 (S1, 3 days); Day 1 iPSCs were first rinsed with PBS without Mg²⁺ and Ca²⁺ and then cultured in the basal medium further supplemented with 0.5% FBS (Thermo, # A3160602), 100 ng/mL Activin A (R&D Systems, Minneapolis, Minn., #338-AC-050) and 3 μM of CHIR-99021 (SelleckChem, Houston, Tex., # S2924). Cells were cultured in 2 mL media each for a well of a 6-well-plate, 1 mL for a 12-well-plate. At day 2, culture supernatants were removed, and cells were incubated with the basal medium with 0.5% FBS and 100 ng/mL Activin A. Cells were further infected with the Lenti-PDX1 vector once at an approximate MOI of 30 on day 2. For the screening of single lentiviral vectors for individual factors, each lentiviral vector was delivered at this time point. At day 3, culture supernatants were replaced by the basal medium with 0.5% FBS and 100 ng/mL Activin A. Step 2 (S2, 2 days); Cells were rinsed with PBS, then cultured with the basal medium 0.5% FBS, 0.25 mM ascorbic acid (Sigma- Aldrich, # A4544) and 50 ng/mL of FGF7 (R&D Systems, #251-KG-050). Culture supernatants were replaced with the same, fresh medium at day 2. Step 3 (S3, 2 days); the culture supernatants were replaced to the basal medium, further supplemented with 14 μL/mL of 7.5% sodium bicarbonate solution, 2% FBS, 0.25 mM ascorbic acid, 50 ng/mL FGF7, 0.25 μM SANT-1 (Sigma-Aldrich, # S4572), 1 μM retinoic acid (Sigma-Aldrich, # R2625), 100 nM LDN193189 (Stemgent, Lexington, Mass., #04-0019), 1:200 ITS-X (Thermo Fisher Scientific, #51500056), and 100 nM alpha amyloid protein modulator (also called TPB, EMD Millipore, Billerica, Mass., #565740). Medium was changed every day. Step 4 (S4, 3 days); the culture supernatants of S3 cells were replaced with the basal medium further supplemented with 14 μL/mL of 7.5% sodium bicarbonate solution, 2% FBS, 0.25 mM ascorbic acid, 2 ng/mL of FGF7, 0.25 μM SANT-1, 0.1 μM retinoic acid, 200 nM LDN193189, 1:200 ITS-X, and 100 nM TPB. Cells also were infected with the Lenti-NEUROG3 vector at an approximate MOI of 30 once on day 7 (end of Step 3) or day 8 (beginning of Step 4). Medium changed every day with the fresh media. Step 5 (S5, 3 days); cells were cultured in the basal medium further supplemented with 14 μL/mL of 7.5% sodium bicarbonate solution, 4 μL/mL of 45% glucose solution, 2% FBS, 0.25 μM SANT-1, 10 μM ALK5 inhibitor II (Enzo Life Sciences, Farmingdale, N.Y., # ALX-270-445), 0.05 μM retinoic acid, 1 μM thyroid hormone (Sigma-Aldrich, # T6397), 100 nM LDN193189, 1:200 ITS-X, 10 μM zinc sulfate (Sigma-Aldrich, # Z0251) and 10 μg/mL heparin (Sigma-Aldrich, # H3149). Medium was replaced every day. Step 6 (S6, 7 days); culture media were replaced with the basal medium further supplemented with 14 μL/mL of 7.5% sodium bicarbonate solution, 4 μL/mL of 45% glucose solution, 2% FBS, 1 μM thyroid hormone, 10 μM ALK5 inhibitor II, 10 μM zinc sulfate, 100 nM LDN193189, 1:200 ITS-X, 10 μg/mL heparin, and 100 nM gamma secretase inhibitor XX (EMD Millipore, Billerica, Mass., #565789) for 7 days. Cells also were infected with the Lenti-MAFA vector at an approximate MOI of 30 once, on day 14, 15, or 16. Fresh medium was fed every day.

### qRT-PCR

For quantitative reverse transcription polymerase chain reaction (qRT-PCR), total RNA from differentiated iPSCs at indicated time points was isolated using Trizol according to the manufacturer instructions. cDNAs were then synthesized by reverse transcription from 200 ng of total RNA using SuperScript III Reverse Transcriptase, dNTP solutions, RNaseOUT and Random Hexamer. Hotstart Taq DNA polymerase and primer pairs for human INS, GCK, GLP1R, ESRRG, SLC6A5, SLC30A8, ABCC8, KCNJ11, CACNA1D, CACNA2D3, PCSK1 and PCSK2 were used. Sequence of the primers used for qRT-PCR was listed in Table 1. The PCR conditions were 95° C. for 10 minutes enzyme activation, 95° C. for 15 seconds denaturation, 60° C. for 60 seconds annealing and extension, and overall 40 cycles were performed.

TABLE 1

| | | SEQ | | SEQ |
|---|---|---|---|---|
| Gene | | ID | | ID |
| Name | FWD (5′ to 3′) | NO: | REV (5′ to 3′) | NO: |
| ABCC8 | CGT CTT AGC TGT GCT TCT GT | 15 | CTT GGT CTG TAT TGC TCC TCT C | 16 |
| CACNA1D | GGG AGC AGG AGT ATT TCA GTA G | 17 | GAT GTT TCT GCC TGG GTA TCT | 18 |
| CACNA2D3 | GAT GGC CTC CAA CTG GTA AA | 19 | CAT GTT TCA GGT GTG CTT CTT C | 20 |
| ERRG | TCT CTA CCC TTC TGC TCC TAT C | 21 | GCA TCG AGT TGA GCA TGT ATT C | 22 |
| GAPDH | GCG CCC AAT ACG ACC AA | 23 | CTC TCT GCT CCT CCT GTT C | 24 |
| GCK | GAT GCA CTC AGA GAT GTA GTC G | 25 | TGA AGG TGG GAG AAG GTG AG | 26 |
| GLP1R | CTC CTT CTC TGC TCT GGT TAT C | 27 | CAG GTT CAG GTG GAT GTA GTT | 28 |
| INS | CTT CAC GAG CCC AGC CA | 29 | ATC AGA AGA GGC CAT CAA GC | 30 |
| KCNJ11 | ATG AGG ACC ACA GCC TAC T | 31 | GGA ATC TGG AGA GAT GCT GAA C | 32 |
| PCSK1 | CAG ACA GCA TCT ACA CCA TCT C | 33 | GTG TAA TCT CCG CTG CTG TAA | 34 |
| PCSK2 | GAC CTG GCC TCC AAC TAT AAT G | 35 | TGT GTA CCG AGG GTA AGG ATA G | 36 |
| SLC30A8 | GCA TGC CCT TGG AGA TCT ATT | 37 | GCA GAT TGG GTC GGC TAT TT | 38 |
| SLC6A5 | GAA AGT CTG CTG GGC ATT TG | 39 | CAC CAT GGA CCA GTT AGG ATA G | 40 |

### RNA Sequencing

For RNA sequencing, a total 200 ng RNA from differentiated iPSCs at indicated time points was isolated using RNeasy Mini Kit. Library preparation (TruSeq mRNA v2 (TMRNA)) and next-generation sequencing and analysis (standard secondary analysis pipeline, MAPRSeq) were performed. Heatmaps were depicted using Graphpad Prism.

### Immunofluorescence Staining

Lentiviral vector-infected 293T cells were fixed with 4% paraformaldehyde (PFA) for 20 minures. After fixation, cells were washed once with PBS and were then permeabilized with 0.3% Triton X-100 in PBS for 10 minutes. Cells were then washed with PBS twice and were blocked with 5% FBS in PBS for 1 hour. Cells were incubated overnight with rabbit anti-human PDX1 (1:200, Abcam, Cambridge, Mass., # AB47267), rabbit anti-human NEUROG3 (1:50, DSHB, Iowa City, Iowa, # F25A1B3), rabbit anti-human MAFA (1:200, Abcam, # AB47267), goat anti-human NKX6.1 (1:100, R&D Systems, # AF5857), rabbit anti-human Neurod1(1:200, Sigma, # N3663), mouse anti-human NKX2.2 (1:50, DSHB, #74.5A5), anti-human ESRRG (1:100, Abcam, # AB131593), or mouse anti-human MAFB (1:25, R&D Systems, # MAB3810) overnight at 4° C., followed by incubation with secondary antibodies for 1 hour at room temperature.

For the characterization of psBCs, undifferentiated iPSCs were seeded on chamber slides and subjected to the differentiation protocol described above. Differentiated cells were fixed at the indicated time points and were permeabilized and blocked as described above. Cells were incubated overnight with guinea pig anti-human insulin (1:400, Dako, Santa Clara, Calif., # A056401), goat anti-human NKX6.1 (1:100), rabbit anti-human Neurod1(1:200), mouse anti-human NKX2.2 (1:50), mouse anti-human glucagon (1:300, Abcam, # ab10988-100), rabbit anti-human somatostatin (1:100, Santa Cruz, Dallas, Tex., # sc-20999), or mouse anti-human C-peptide (1:400, Thermo Fisher Scientific, # MA1-19159) overnight at 4° , followed by incubation with secondary antibodies for 1 hour at room temperature.

Mouse kidneys with the grafts were harvested and frozen in OCT Compound. 7 μm pancreatic cryosections were immediately fixed, permeabilized and then blocked as described above. Slides were then incubated with guinea pig anti-human insulin (1:400), goat anti-human NKX6.1 (1:100), rabbit anti-human Neurod1 (1:200), mouse anti-human NKX2.2 (1:50), mouse anti-human glucagon (1:300), or rabbit anti-human somatostatin (1:100) overnight at 4°, followed by secondary antibody incubation for 1 hour at room temperature. Images were taken using a Zeiss LSM 780 confocal laser scanning microscope and analyzed with Zeiss imaging software. Fluorescence intensity were analyzed by Image J software.

### Flow Cytometry

iPSC-derived psBCs were dispersed into single-cell suspension by incubation in Trypsin at 37° C. for 10 minutes and quenched with 3-4 volumes of FCS-containing culture media, and cells were spun down for 5 minutes at 800 g. Cells were transferred to a 1.7 mL microcentrifuge tube, fixed in 4% PFA, permeabilized with 0.3% Triton-X and then blocked with 5% FBS for 1 hour. Cells were incubated overnight with guinea pig anti-human insulin (1:400), mouse anti-human glucagon (1:300), and mouse anti-human C-peptide (1:400) at 4° C. After washing three times with 5% FBS, cells were stained with secondary antibodies. Cell then were washed and filtered through a 35-μm mesh Falcon tube, and analyzed using the LSR-II flow cytometer (BD Biosciences). Analysis of the results was performed using FlowJo software.

### Electron Microscopy

iPSC-derived psBCs were fixed at room temperature. Cell samples were processed and analyzed by transmission Electron Microscopy.

### In vitro GSIS assay

An islet perifusion system (Biorep technologies, Miami Lakes, Fla.) was used. Approximately 1×10⁶ psBCs, differentiated in wells of 48-well plates, were first incubated in 4 mM glucose Krebs Ringer Bicarbonate buffer supplemented with 0.2% BSA for 30 minutes at 37° C. Cells were then gently scraped off from the well, transferred into a sterile 1.5 mL eppendorf tube, and centrifuged at room temperature for 5 minutes. Cell pellets were transferred into the perifusion chamber and were washed for 40 minutes in the perfusion system with 4 mM glucose buffer, which were preheated to 37° C. and oxygenized with 95% O₂ and 5% CO₂. After washing, cells were exposed to 4 mM glucose perifusate for 32 minutes, followed by 32 minutes of 16 mM glucose buffer, 32 minutes of 16 mM glucose buffer supplemented with 100 nM GLP-1 (Peprotech, Rocky Hill, N.J., #130-08-1MG), 32 minutes of 4 mM glucose buffer and finally 8 minutes of 16 mM glucose buffer supplemented with 30 mM KCL. Effluent was collected in 2-minute intervals and assayed for human C-peptide by ELISA (Alpco, Salem, N.H., #80-CPTHU-CH01) for subsequent determination of basal and GSIS. Stepwise PNM transduction study was performed in triplicate.

### Mice Transplantation Studies

Immunodeficient Fox Chase SCID-Beige mice, aged 8-10 weeks, were purchased from Charles River Laboratory. To induce diabetes, mice received 50 mg/kg body weight streptozotocin (STZ, Sigma, # S0130) intraperitoneally over the course of five consecutive days. Mice with nonfasting blood glucose levels over 250 mg/dL after STZ administration were used and randomized into four groups in the following experiment. PsBCs clusters (approximately 50 million cells per mouse), and human beta-cell line (EndoC-βH2 cells, approximately 5 million cells per mouse) were gently scraped off or trypsinized and transferred into a 15 mL conical vial. After spinning for 5 minutes at 800 g, cells were loaded into a catheter for cell delivery into the kidney capsules (E l Khatib et al., Gene Ther., 22:430-438 (2015)). 1 day, 4 day, and 1, 2, 3, 5, 7, 9, 11, and 13 weeks after the surgery, fasting (16 hours) blood glucose was tested using glucose monitor and strip. To measure glucose-responsive C-peptide secreion, fasting blood and 30 minutes blood after an intraperitoneal injection of D-(+)-glucose at 2 g/kg body weight was collected through retro orbital bleeding every two weeks. Serum was separated out using Microvettes (Sarstedt, Nümbrecht, Germany, #20.1278.100) and stored at 80° C. until ELISA analysis. At indicated time points, kidneys containing the grafts were dissected from the mice, embedded and frozen in OCT compound. Immunostaining was performed as described above. No statistical method was used for sample size estimation. Investigators were not blinded to the group allocations. Mice transplantation study was performed in duplicate.

### Intraperitoneal Glucose Tolerance Test (IPGTT)

To measure glucose handling capacity in vivo, mice were fasted (16 hours), and blood glucose was tested at 0 minutes, 30 minutes, 60 minutes, 90 minutes, and 120 minutes after IP injection of D-(+)-glucose at 2 g/kg body weight.

### Sample Size and Statistical Analysis

All data represent the means±S.E.M. of three to nine samples, as indicated in the Figure legends for the Figures of Example 2. Group comparisons were analyzed by unpaired or paired t tests, one sample t test, and one-way ANOVA with Tukey test through IBM SPSS Statistics 22. Bar graphs, heatmaps, curves, box and whisker plots were generated with GraphPad Prism? and Excel 2010.

### Results

### Screening of Beta-Cell Transcription Factor(s) for Improved Glucose- and GLP-1-Responsive Insulin Secretion in psBCs

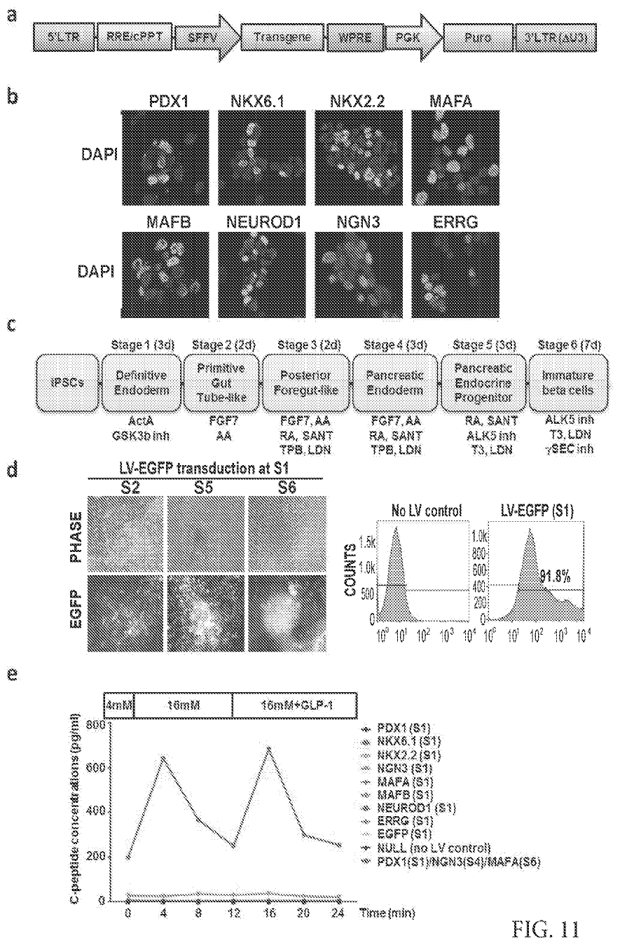

Lentiviral vectors carrying codon-optimized ORFs of transcription factors critical for beta-cell development and function, including PDX1, NKX6.1, NKX2.2, MAFA, MAFB, NEUROD1, NEUROG3 and ESRRG (FIG. 11A), were produced. Vector titers were determined by puromycin resistance, and the expression of encoded transgene proteins was verified in vector-infected 293T cells by immunostaining with specific antibodies (FIG. 11B). Monolayer iPSCs underwent a guided differentiation process for 3 weeks (FIG. 11C). When differentiating iPSC progeny at Stage 1 (S1, Day 2) was transduced by a control EGFP-expressing lentiviral vector at an approximate multiplicity of infection (MOI) of 30, EGFP signals were found throughout the differentiation process from S2 to S6 (FIG. 11D, left panel). Flow cytometry analysis demonstrated that over 90% of cells were EGFP-positive at the end of S6 stage (FIG. 11D, right panel). Efficient EGFP transduction was also found when iPSC progeny was transduced at other stages.

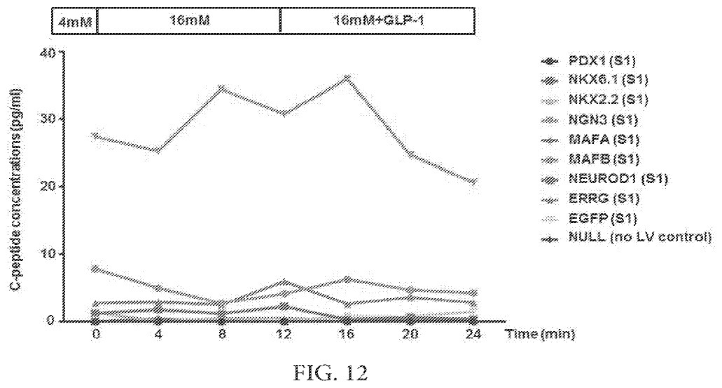

To determine whether the introduction of a single, key beta-cell transcription factor could improve the glucose-responsiveness of psBCs, Si iPSC progeny were first transduced with a single lentiviral vector carrying a beta-cell factor. Since over-expression of PDX1, NEUROG3 and MAFA has been shown to transdifferentiate liver and pancreatic exocrine cells into insulin-producing cells (Zhu et al., Stem Cell Res. Ther., 8:64 (2017)), iPSC progeny also were transduced with a combination of lentiviral vectors expressing the PDX1, NEUROG3 and MAFA triad, at Stages 1, 4 and 6, respectively. Perifusion experiments of S6 psBCs demonstrated very low level, no glucose-responsive C-peptide secretion by unmodified (NULL) or EGFP vector-infected control cells (FIG. 11E). Introduction of PDX1, NKX6.1, NKX2.2, MAFA, MAFB, NEUROD1, or ESRRG at stage 1 did not strongly affect C-peptide secretion or glucose-responsiveness. In contrast, introduction of NEUROG3 alone strongly enhanced the insulin secretory capacity of resulting psBCs, from up to 0.08 pg/mL of C-peptide secretion in unmodified control psBCs and up to 36 pg/mL in NEUROG3-transduced psBCs (FIG. 12). Nevertheless, NEUROG3 transduction alone did not improve glucose-responsiveness of resulting psBCs. Notably, stepwise transduction of PDX1, NEUROG3, and MAFA together (PNM) in differentiating iPSC progeny led within 3 weeks to the generation of psBCs with notable glucose- and GLP-1-responsiveness, along with increased insulin secretory capacity.

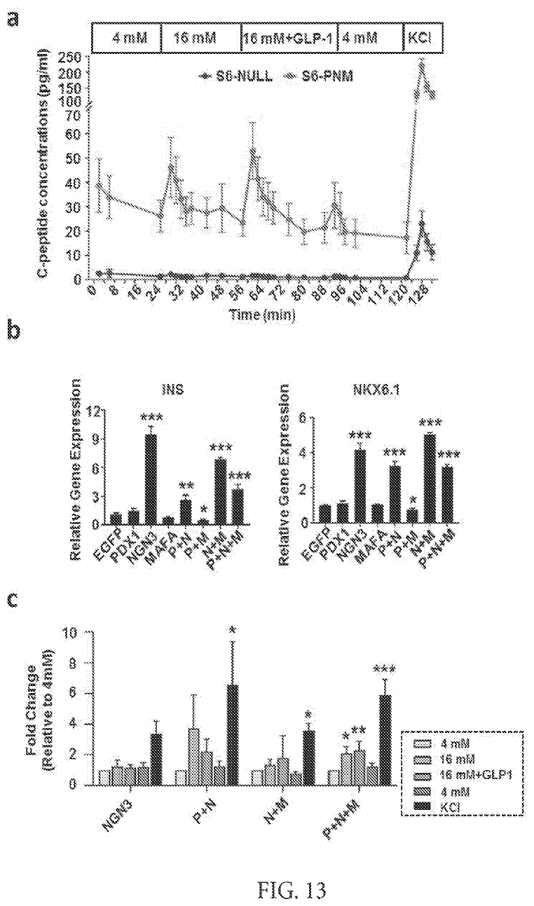

Stepwise PDX1, NEUROG3, and MAFA transduction facilitated generation of glucose- and GLP-1-responsive psBCs. To assess the reproducibility of generation of glucose- and GLP-1-responsive psBCs by PNM transduction, the dynamics of C-peptide secretion from S6 psBCs with or without PNM transduction were analyzed. Rapid responses were observed in C-peptide secretion from PNM transduced S6 psBCs upon sequential increases of glucose levels from 4 to 16 mM, addition of GLP-1 to 16 mM glucose, and in 30 mM KCl depolarization (FIG. 13A). When the fold changes (FC) were assessed within individual samples at the medium-changing time points, a 2.1 fold increase was found in 16 mM glucose stimulation at the 4-min time point (P=0.03), when compared to the level at the last time point in the previous stage. Immediate 2.2 fold increase in 16 mM glucose+GLP-1 (P<0.001), no significant change on reversion to 4 mM (P=0.146), and 10.7 fold increase in KCl treatment (P<0.001) were also found. Additionally, one feature of immature fetal beta cells is the exaggerated insulin secretion under low glucose (Blum et al., Nat. Biotechnol., 30:261-264 (2012)). However, repressed C-peptide secretion from PNM transduced S6 psBCs in basal 4 mM glucose treatment was observed. Moreover, PNM transduced S6 psBCs also displayed a clear 1^{st} and 2^{nd} phase insulin response upon 16 mM glucose treatment, which was another feature of mature beta cells (Pagliuca et al., Cell, 159:428-439 (2014); and Song et al., J. Clin. Endocrinol. Metab., 85:4491-4499 (2000)). Together, these results suggest that PNM transduction promotes the differentiation of iPSCs to glucose-responsive, incretin (GLP-1)-responsive mature beta cells in vitro.

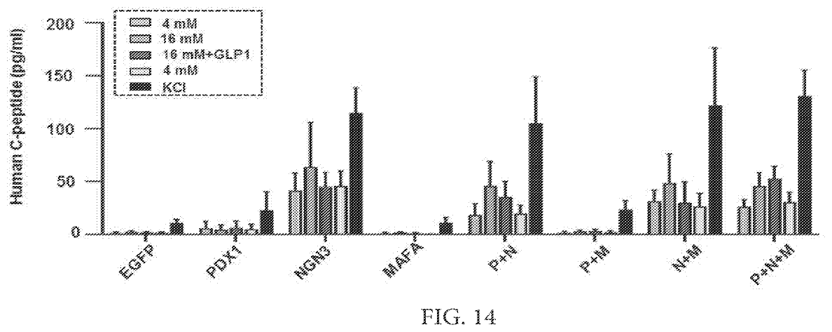

To further assess the contributions of PDX1 (Stage 1), NGN3 (Stage 4), and MAFA (Stage 6), the impact of transduction of different combinations of P/N/M was determined. All psBC that include NGN3 (NGN3 alone, PDX1+NGN3, NGN3+MAFA, and PDX1+NGN3+MAFA) exhibited increased levels of INS and NKX6.1 transcripts (FIG. 13B). Transduction with PDX1 alone, MAFA alone, or both PDX1 and MAFA exhibited no significant effect (FIG. 13B). When the GSIS of psBCs was assessed by the perifusion system, groups with NEUROG3 transduction exhibited higher baseline C-peptide secretion as well as higher total secretable C-peptide contents, whereas psBCs groups without NEUROG3 transduction exhibited little C-peptide secretion (FIG. 14). Among the high C-peptide releasing groups by NEUROG3 transduction, only the PNM transduction group displayed significantly higher C-peptide levels at 16 mM (P=0.033), 16 mM+GLP-1 (P=0.005), and upon KCl depolarization (P<0.001) relative to the 4 mM glucose baseline (FIG. 13C). psBCs transduced with both PDX1 and NEUROG3 exhibted a trend of enhanced C-peptide secretion upon high glucose stimulation, whereas transduction with NEUROG3 alone or MAFA+NEUROG3 led to no significant change in glucose-responsive C-peptide secretion. These results indicated that NEUROG3 transduction is involved in generating insulin-secreting beta-like cells from iPSCs, while additional PDX1 and MAFA transduction are involved in inducing glucose-responsiveness in psBCs.

### Mature Beta-Cell Characteristics Found in PNM Transduced psBCs

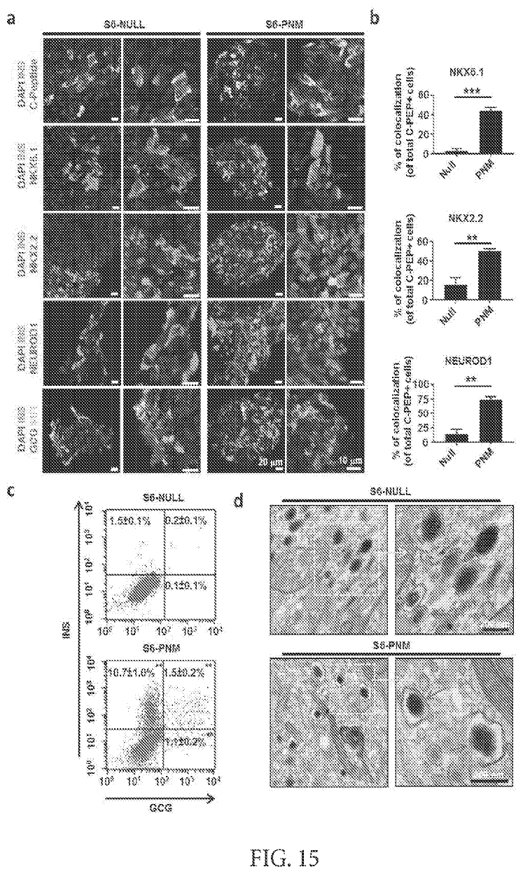

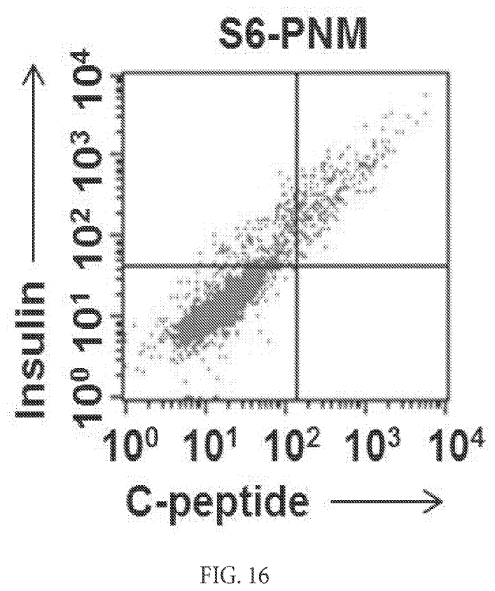

The expression of beta-cell markers in PNM-transduced (S6-PNM) and control (S6-NULL) psBCs were characterized. Immunohistochemistry revealed that C-peptide signals were frequently co-localized with insulin (INS) signals in both S6-PNM and S6-NULL psBCs (FIG. 15A). Pancreatic α-cell marker glucagon (GCG) and δ-cell marker somatostatin (SST) signals also were found, with some multi-hormonal psBCs in both groups (FIG. 15A). In contrast, widespread expression of NKX6.1, NKX2.2, and NEUROD1 was only seen in S6-PNM psBCs (FIG. 15A). This resulted in more frequent co-expression of NKX6.1 (P<0.001), NKX2.2 (P=0.006), and NEUROD1 (P=0.004) in insulin-positive cells in S6-PNM compared to S6-NULL psBCs (FIG. 15B). These observations indicated that stepwise PNM transduction promoted the induction of beta-cell transcription factors NKX6.1, NKX2.2, and NEUROD1 in insulin-producing cells. Flow cytometry analysis revealed that S6-PNM populations contained 12.2% insulin-positive cells and 2.6% glucagon-positive cells, compared to 1.7% and 0.3% in S6-NULL cells, respectively (FIG. 15C). Additionally, insulin-positive cells also were positive for human C-peptide (FIG. 16), further confirming endogenous insulin synthesis rather than insulin uptake from culture media in psBCs.

Transmission electron microscopy analysis was performed to characterize ultra-structures of secretory granules in S6-NULL and S6-PNM psBCs (FIG. 15D). Consistent with flow cytometry analysis, more cells were observed with endocrine granules in S6-PNM psBCs than in S6-NULL psBCs. In S6-PNM psBCs, there were numerous insulin granules with characteristic electron-dense crystal cores surrounded by a light halo, similar to normal human beta-cell insulin granules (Deconinck et al., Diabetologia, 8:326-333 (1972)). In contrast, S6-NULL psBCs often presented both insulin granules as well as glucagon granules characterized by larger dense cores and greyer halo (Deconinck et al., Diabetologia, 8:326-333 (1972)). These results further support the improved maturation pattern of psBCs upon stepwise PNM transduction.

### Global Gene Expression Profiling Identifies Accelerated Induction of Glucose Sensing and Insulin Secretion Genes Upon PNM Transduction

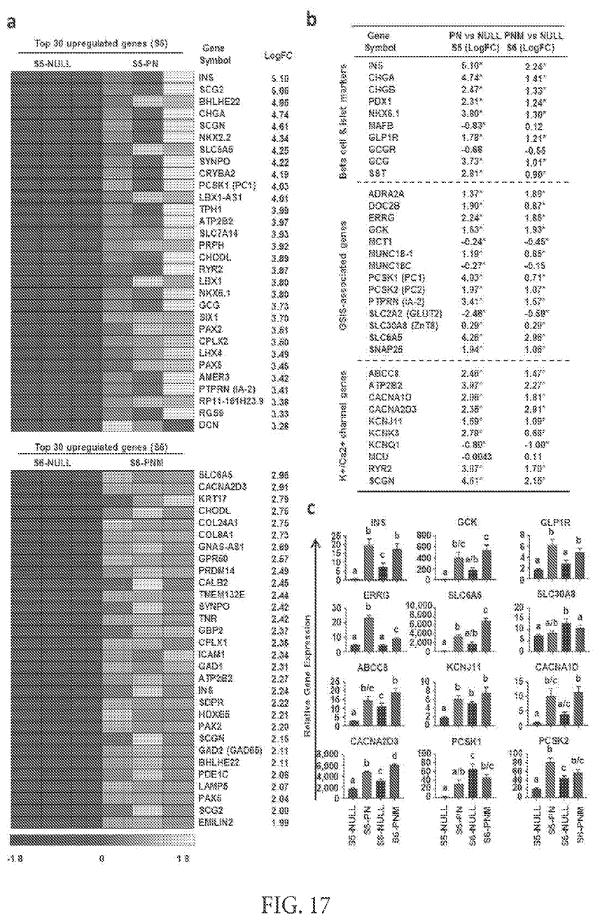

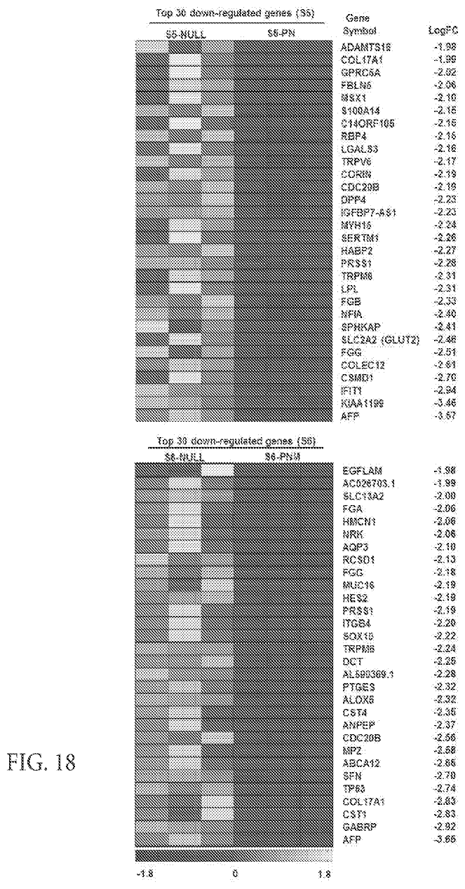

To further understand the impact of PNM transduction on iPSC differentiation into psBCs, next generation RNAseq analyses were performed using RNA samples from psBCs at the end of Stage 5 (control S5-NULL cells vs. S5-PN cells, 13 days after differentiation, before S6 MAFA transduction) as well as psBCs at the end of Stage 6 (control S6-NULL cells vs. S6-PNM, 20 days after differentiation). The top 30 up-regulated genes for S5-PN and S6-PNM identified key genes relevant to beta-cell development and function including INS, NKX2.2, NKX6.1, PAX2, PCSK1 (PC1), as well as insulin granule exocytosis, such as CHGA, SCG2/CHGC, SCGN, CPLX1 and CPLX2. The major type 1 diabetes antigen genes, including INS, IA-2 (PTPRN) and GAD65 (GAD2), also were prioritized. Other notable genes identified in both S5 and S6 stages included SLC6A5/GLYT2, which controls a glycine-insulin autocrine feedback (Yan-Do et al., Diabetes, 65:2311-2321 (2016)), BHLHE22 regulating insulin gene expression (Melkman-Zehavi et al., EMBO J., 30:835-845 (2011)) and a plasma membrane Ca²⁺-ATPase, ATP2B2/PMCA2, which affects GSIS and beta-cell proliferation (Pachera et al., Diabetologia, 58:2843-2850 (2015)) (FIG. 17A). Further analysis of sets of genes, associated with beta-cell/islet maturation, GSIS, L-type voltage-dependent calcium channel (VDCC) and K_{ATP} channels, also identified significant upregulation of genes underlying beta-cell functionality, including GCK, ESRRG, CHGB, SLC30A8, ABCC8, KCNJ 11 and CACNA2D3 (FIG. 17B). In addition, GCG and SST expression also were upregulated, especially at S5, suggesting accelerated endocrine differentiation by PDX1 and NEUROG3 transduction. Quantitative RT-PCR analysis verified upregulation of key beta-cell factors in S5-PN and S6-PNM psBCs (FIG. 17C). Of note, when top 30 down-regulated genes were identified for S5-PN and S6-PNM (FIG. 18), a hepatic progenitor marker AFP was found as the most prominently suppressed gene upon PNM transduction, suggesting directed beta-cell specification by PNM transduction in derived iPSC progeny.

### PNM-Modified psBCs Demonstrate Glucose-Responsive Insulin Secretion In Vivo

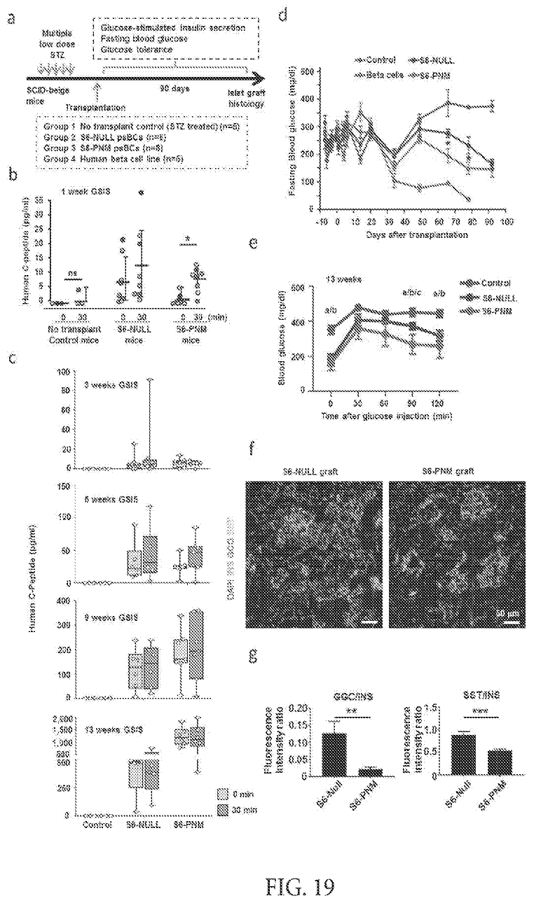

To evaluate the glucose-responsive insulin secretory capacity of PNM-modified psBCs in vivo, approximately 50 million S6 psBCs were transplanted in the kidney capsules of immunodeficient SCID-beige mice with STZ-induced diabetes (FIG. 19A). A human beta-cell line also was transplanted as a control. When glucose-responsive insulin secretion was monitored upon intraperitoneal glucose administration, low levels of human C-peptide were found in circulation in S6-NULL and S6-PNM recipient mice 1 week after transplantation. Importantly, levels of circulating human C-peptide were significantly increased 30 minutes after glucose challenge in S6-PNM recipient mice (P=0.003) (FIG. 19B), although S6-NULL recipient mice did not show in vivo glucose-responsiveness. No notable circulating human C-peptide was seen in control mice, while mice transplanted with the human beta-cell line showed no glucose-responsive, but higher levels of human C-peptide before (average 15.1 pg/mL) and after (average 18.4 pg/mL) glucose challenge. These results demonstrated the glucose-responsive insulin secretory capacity of PNM-transduced psBCs in vivo, as early as 1 week post transplantation.

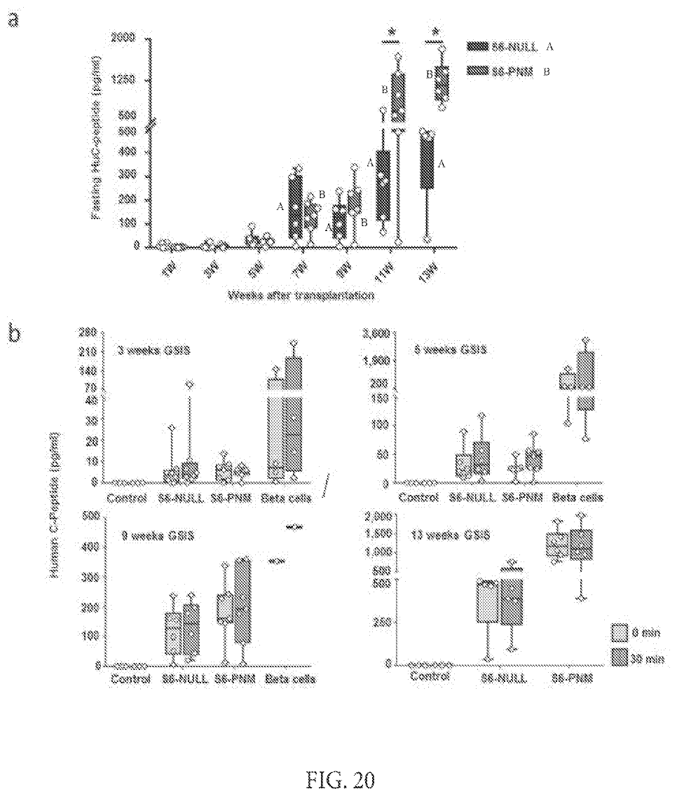

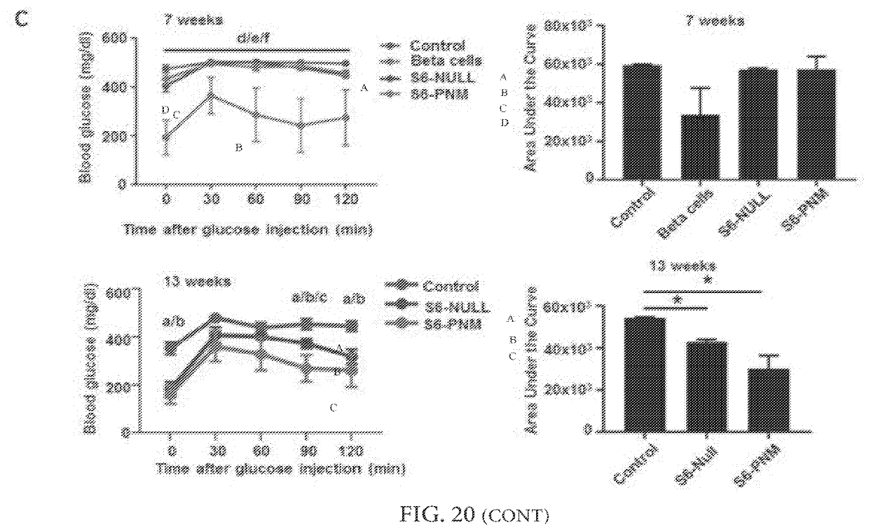

Fasting human C-peptide levels in S6-PNM-transplanted mice gradually increased overtime, from 3 pg/mL at 1 week to 1,172 pg/mL at 13 weeks post transplantation (FIG. 20A). GSIS was not detected at 3, 5, 9, and 13 weeks (FIGS. 19C and 20B). Similar results were found in S6-NULL-transplanted mice; however, the circulating C-peptide levels were significantly lower than those observed in S6-PNM at weeks 11 (P=0.047) and 13 (P=0.048) (FIGS. 19C and 20A). Similarly, although blood glucose levels after 16 hours of fasting remained high in both S6-NULL and S6-PNM psBCs-transplanted mice for 3 weeks, S6-NULL- and S6-PNM-recipient mice started to exhibit lower blood glucose levels than controls at 8 weeks post-transplant (FIG. 19D). The blood glucose levels were significantly lower in S6-PNM mice than those in S6-NULL mice at 9 (P=0.022) and 11 (P=0.047) weeks post transplantation (FIG. 19D). To assess the impact of psBC transplantation on the glucose tolerance in vivo, IPGTT also was conducted at 7 and 13 weeks post-transplant. There was no difference observed among diabetic controls, S6-NULL- and S6-PNM-recipient mice in glucose level at 7 weeks (FIG. 20C). However, at 13 weeks, S6-PNM recipient mice displayed lower glucose levels than S6-NULL at 90 minutes (P=0.003), indicating superior glucose regulation by S6-PNM than S6-NULL psBCs (FIGS. 19E and 20C).

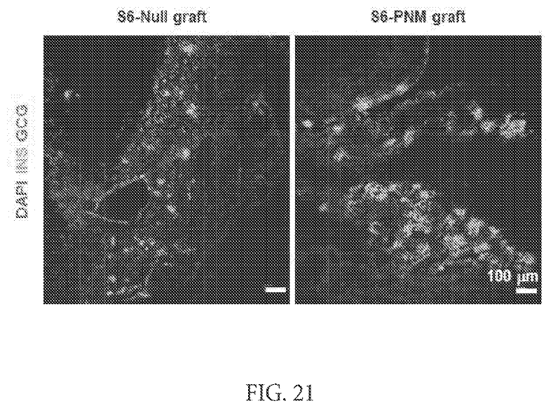

Prospectively, 13 weeks after transplantation, the kidneys with psBC grafts were harvested from the recipient mice. Further analysis revealed that the insulin-positive cells in S6-PNM and S6-NULL grafts were largely monohormonal. However, S6-PNM grafts contained rich clusters of insulin-positive cells, whereas in S6-NULL grafts the insulin-positive cells were scattered (FIGS. 19F and 21). Notably, insulin-positive cells in S6-NULL were accompanied by significantly more glucagon-positive (P=0.007) and somatostatin-positive cells (P<0.001) compared to S6-PNM (FIGS. 19F-G), suggesting that the less mature S6-NULL cells may further adopted alpha cell and delta cell destiny in vivo.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A population of differentiated cells obtained from embryonic stem cells or induced pluripotent stem cells, wherein the cells of said population produce insulin in response to glucose and in response to GLP-1 as measured by a perifusion assay.

2. The population of differentiated cells of claim 1, wherein said cells are human cells.

3. The population of differentiated cells of claim 1, wherein said cells lack exogenous nucleic acid.

4. The population of differentiated cells of claim 1, wherein said cells produce insulin in less than five minutes (e.g., in less than four, three, two, or one minute) in response to at least 16 mM of glucose and in response to at least 100 nM of GLP-1 as measured by a perifusion assay.

5. A method for obtaining a population of differentiated cells obtained from embryonic stem cells or induced pluripotent stem cells, wherein said differentiated cells produce insulin in response to glucose or GLP-1, wherein said method comprises:
(a) introducing a vector comprising nucleic acid encoding a PDX1 polypeptide into differentiating cells of an embryonic stem cell population or of an induced pluripotent stem cell population at or during the definitive endoderm stage to form a first cell population,
(b) introducing a vector comprising nucleic acid encoding an NGN3 polypeptide into cells of said first cell population at or during the pancreatic endoderm stage to form a second cell population, and
(c) introducing a vector comprising nucleic acid encoding a MAFA polypeptide into cells of said second cell population at or during the primitive beta cell stage to form said population of differentiated cells.

6. The method of claim 5, wherein said induced pluripotent stem cells are human induced pluripotent stem cells.

7. The method of claim 5, wherein said differentiated cells produce insulin in response to high glucose or GLP-1 as measured by a perifusion assay.

8. The method of claim 5, wherein said vector of said step (a), (b), or (c) is a lentiviral vector.

9. The method of claim 5, wherein said vector of said step (a), (b), and (c) is a lentiviral vector.

10. The method of claim 5, wherein the total number of days starting with said induced pluripotent stem cells to forming said population of differentiated cells is 20 or less days.
